(19) United States
(12) Patent Application Publication
(10) Pub. No.: US 2023/0329689 A1
(43) Pub. Date: Oct. 19, 2023

(54) TISSUE PIERCING ASSEMBLIES AND METHODS OF USE

(71) Applicant: SHIFAMED HOLDINGS, LLC, Campbell, CA (US)

(72) Inventors: Tom SAUL, Portland, OR (US); Joann HEBERER, Portola Valley, CA (US)

(21) Appl. No.: 18/245,800

(22) PCT Filed: Sep. 17, 2021
(86) PCT No.: PCT/US2021/050787

§ 371 Date: Mar. 17, 2023

Related U.S. Application Data
(60) Provisional application No. 63/079,847, filed on Sep. 17, 2020.

(51) Int. Cl. A61B 17/00; A61B 17/34

(52) U.S. Cl. CPC A61B 17/0057; A61B 17/00234; A61B 17/34; A61B 2017/00243; A61B 2017/3484; A61B 2017/00597

(57) ABSTRACT

Tissue piercing devices and methods for using the same are described. The tissue piercing devices may be adapted to pierce through the septal wall to create a pathway between the right and left atria. The tissue piercing devices may include one or more visualization elements to visualize a target puncture site on the septal wall, such as within the fossa ovalis. The devices that may include an inner volume adapted to hold a piercing member and the one or more visualization elements therein. One or more components of the tissue piercing apparatus may be steerable to steer the piercing member toward the target puncture site. In some examples, the tissue piercing devices may include an inflatable member that is expandable upon inflation to form the inner volume.

### CROSS REFERENCE TO RELATED APPLICATIONS

This application claims priority to U.S. Provisional Application No. 63/079,847, entitled “TISSUE PIERCING ASSEMBLIES AND METHODS OF USE,” and filed on Sep. 17, 2020, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

The fossa ovalis (“FO”) is a depression in the right atrium of the heart, at the level of the interatrial septum, the wall between right and left atrium. Some existing medical procedures include piercing a FO of an interatrial septum to create an opening and pathway between right and left atria. This opening can facilitate passage of one or more devices between the atria of the heart. Creating this pathway provides more access pathway options for medical procedures. For example only, minimally invasive medical procedures related to a left atrium, mitral valve (e.g., mitral valve repair, replacement mitral valve), left atrial appendage, pulmonary artery, left atrial mapping, left atrial ablation, may be performed by advancing one or more therapeutic and/or diagnostic tools from a right atrium into a left atrium through an opening created in a FO. By way of example only, access to a right atrium may be obtained through a superior vena cava (“SVC”) or an inferior vena cava (“IVC”), examples of which are known.

Some existing procedures for piercing a FO include delivering a catheter into the right atrium (e.g., from an IVC) urging the catheter against the FO, and then advancing a needle from the catheter through the FO to create the opening. Existing procedures generally rely on X-ray and Transesophageal Echocardiography (“TEE”) ultrasound imaging to provide visualization for proper catheter placement. The FO is a very thin membrane, and as the catheter is pushed against the FO, tenting of the FO can generally be seen on the image. Additionally, the physician may attempt to feel when the catheter is placed against the very thin FO to help determine proper catheter positioning. These techniques are less than optimal, and it may still prove difficult to determine that the catheter is in an appropriate or optimal position against the FO before advancing the needle. For example, if the catheter is not positioned adequately, the needle may receive more resistance from thicker tissue adjacent the FO when attempting to pierce the tissue. This may increase the likelihood of pushing too hard with the needle against the tissue, and when the needle pierces the tissue and the resistance drops quickly, the force being applied with the needle may accidentally push the needle into left atrial tissue, such as the left atrium wall or left atrial appendage tissue, damaging cardiac tissue.

Additionally, in some cases the piercing technique is a specialized technique, and some physicians may refuse to perform procedures that requires a transseptal puncture. Additionally, a physician performing a subsequent procedure (e.g., left atrial ablation, mitral valve replacement) may be reluctant or unable to perform the piercing procedure, and a separate physician may be required to perform the piercing procedure.

There is a need for improved procedures for piercing a FO, which may address one or more of the deficiencies set forth above.

### SUMMARY

Described herein are tissue piercing devices and related devices and systems. The tissue piercing devices may be adapted to pierce through areas of the atrial septal wall (e.g., FO) to create a pathway between the right and left atria. The devices may include one or more optical elements that are used to visualize a target puncture site on the septal wall. The devices may include an inner volume adapted to hold a piercing member (e.g., needle). The tissue piercing devices may include an inflatable member that has expandable walls that expand upon inflation to form the inner volume. In some examples, the tissue piercing devices may include at least two inflatable volumes: an outer volume (also referred to herein a second volume or second internal volume) within the wall of the inflatable member, and an inner volume (also referred to herein as a first volume or first internal volume) defined by a cavity that the inflatable member forms when inflated. For example, for a cone-shaped inflatable member, the outer volume may refer to the volume within the walls of the cone, and the inner volume may refer to the volume within the cone. In some examples the one or more components of the tissue piercing device are steerable to position the device relative to the septal wall and/or to steer the piercing member toward the target puncture site.

One aspect of the disclosure is a tissue piercing assembly configured to pierce a septal wall between a right atrium and a left atrium, comprising: a cone-shaped member having inflatable walls and a closed inner chamber, wherein a closed distal region of the inner chamber includes a membrane; a visualization member adapted to be positioned to have a field of view that includes at least a portion of the closed distal region; and one or more of a piercing member guide and a piercing member disposed within the inflatable chamber, wherein the piercing member has a distal end that is configured to pierce the membrane at the closed distal region of the cone-shaped member.

In this aspect, the inflatable wall may comprise one or more second inflatable chambers disposed radially outward relative to a proximal region of the inflatable chamber, at least one of the one or more second inflatable chambers not in direct fluid communication with the closed inner chamber.

In this aspect, the membrane may have a lesser thickness than the inflatable walls. In this aspect, the closed distal region is adapted to have a planar or flattened configuration when the cone-shaped member is at least partially inflated.

In this aspect, the closed distal region may be less stiff than a proximal region of the cone-shaped member when the cone-shaped member is at least partially inflated.

In this aspect, the proximal region may include at least one second inflatable chamber.

In this aspect, the assembly may further comprise one or more controllers configured to control a first fluid pressure within the closed inner chamber and a second fluid pressure within the inflatable walls, wherein the first fluid pressure is different than the second fluid pressure.

In this aspect, the closed distal region may have a flattened configuration when the cone-shaped member is in an at least partially inflated configuration.

In this aspect, the assembly may include the piercing member guide and the piercing member, the piercing member guide including a piercing member lumen sized and configured to receive the piercing member therethrough.

In this aspect, the one or more of the piercing member guide and the piercing member may be axially movable relative to the cone-shaped member.

In this aspect, the assembly may include the piercing member guide, the piercing member guide having a sharpened distal end.

In this aspect, the assembly may include the piercing member guide, the piercing member guide not having a sharpened distal end that it is not configured to pierce tissue.

In this aspect, the visualization member may include an optical element secured relative to the one or more of the piercing member guide and the piercing member that is disposed within the cone-shaped chamber.

In this aspect, the optical element may be secured relative to the one or more of the piercing member guide and the piercing member such that the optical element is adapted to self-deploy to a deployed configuration.

In this aspect, the optical element may be coupled to an arm, the arm secured to the one or more of the piercing member guide and the piercing member, the arm configured to self-deploy to thereby cause the optical element to self-deploy.

In this aspect, the assembly may include the piercing member guide, wherein the visualization member is disposed within a visualization member receiving area of the piercing member guide when in a delivery state.

In this aspect, the visualization member receiving area may be a recessed region of the piercing member guide.

In this aspect, the visualization member may include one or more optical elements secured to the cone-shaped member.

In this aspect, the one or more optical elements may be secured to an inner surface of the closed inner chamber.

In this aspect, the one or more optical elements may be secured to one or more stiffening elements that are secured to an inner surface of cone-shaped member.

In this aspect, the assembly may further comprise electronics secured to the one or more optic elements, the electronics secured to the inner surface of the closed inner chamber.

In this aspect, the electronics may include at least one conductive element.

In this aspect, the visualization member may be secured within any of the one or more second inflatable chambers.

In this aspect, the visualization member may comprise first and second optical elements that are axially spaced apart relative to the cone-shaped member.

In this aspect, the assembly may further comprise one or more coupling members that are configured to couple to one or more secondary medical tools.

In this aspect, the one or more secondary medical tools may include a visualization tool. In this aspect, the visualization member may comprise one or more cameras.

In this aspect, the visualization member may comprise one or more LEDs.

In this aspect, the assembly may further comprise an elongate member secured to and extending proximally from the cone-shaped member.

In this aspect, the elongate member may be steerable.

In this aspect, the assembly may include the piercing member guide, wherein the piercing member guide is steerable.

In this aspect, the visualization member may be steerable, either directly, or indirectly by steering a component to which the visualization member is coupled.

One aspect of the disclosure is a method of piercing a fossa ovalis, comprising: delivering a cone-shaped member having a closed distal region into a right atrium while the cone-shaped member is in a contracted configuration, the closed distal region comprising a membrane; at least partially inflating an inflatable wall of the cone-shaped member to transition the cone-shaped member to an expanded configuration in the right atrium; at least partially inflating a closed inner chamber of the cone-shaped member in the right atrium; providing a field of view that includes the membrane and the fossa ovalis with a visualization member disposed within the closed inner chamber of the cone-shaped member; and piercing the membrane of the cone-shaped member and the fossa ovalis with a piercing member to create an opening in the fossa ovalis.

In this aspect, piercing the membrane and the fossa ovalis may comprise advancing a piercing member through the membrane and the fossa ovalis.

In this aspect, piercing the membrane may comprise activating a RF electrode.

In this aspect, the method may further comprise advancing a piercing member guide distally and into contact with an inner surface of the membrane.

In this aspect, method may further comprise distending the membrane towards the fossa ovalis.

In this aspect, distending the membrane towards the fossa ovalis may comprise pushing on the membrane from within the closed inner chamber.

In this aspect, distending the membrane towards the fossa ovalis may comprise increasing fluid pressure within the cone-shaped member.

In this aspect, method may further comprise tensioning the fossa ovalis when the fossa ovalis is in contact with the membrane.

In this aspect, method may further comprise generating an image of the patient with the secondary medical tool after coupling the secondary medical tool to the elongate member.

One aspect of the disclosure is a tissue piercing device configured to pierce a septal wall, comprising: an inflatable member adapted to assume a conical shape when walls of the inflatable member are inflated with a fluid, wherein the conical shape defines an inner volume and a distal annular rim, the distal annular rim having an engagement surface configured to engage with the septal wall; and a piercing member disposed within the inner volume, the piercing member having a distal end that is adapted to pierce through the septal wall.

In this aspect, the device may further comprise a visualization member adapted to be positioned within the inner volume and to have a field of view through a distal opening defined by the distal annular rim.

In this aspect, the inner volume may be adapted to hold a negative pressure when the distal annular rim is sealed against the septal wall.

In this aspect, the inflatable member may be adapted to assume a contracted configuration when fluid pressure within the walls is below a threshold fluid pressure and the inflatable member is retracted within a delivery catheter.

In this aspect, the inner volume may be configured to be maintained at a first pressure, and the walls are configured to be maintained at a second pressure, wherein the first pressure is different than the second pressure.

In this aspect, the device may further comprise an elongate member, wherein the inflatable member is positioned at a distal end of the elongate member.

In this aspect, the elongate member may include at least one fluid channel adapted to supply fluid to inflate the walls.

In this aspect, the elongate member may include at least one negative pressure channel adapted to supply negative pressure to the inner volume.

In this aspect, the elongate member may be steerable.

In this aspect, the inflatable member may be adapted to communicate with one or more controllers configured to control pressure within one or both of the inner volume and the walls.

In this aspect, the piercing member may include an RF electrode adapted to piercing the septal wall when activated.

In this aspect, the piercing member may be configured to translate distally toward a distal end of the inflatable member to pierce the septal wall.

In this aspect, the piercing member may be configured to translate within a piercing member guide.

In this aspect, the piercing member guide may be configured to translate distally within the inner volume of the inflatable member.

In this aspect, the piercing member guide may be steerable.

In this aspect, the device may further comprise a visualization member positioned within the inner volume, the visualization member being steerable, either directly or indirectly, by steering a component to which the visualization member is coupled.

In this aspect, the visualization member may comprise one or more cameras.

In this aspect, the visualization member may comprise one or more LEDs.

One aspect of the disclosure is a method of piercing a fossa ovalis, comprising: inflating an inflatable member within the right atrium, wherein when inflated, the inflatable member comprises an inner volume and open distal region defined by a distal rim; engaging the distal rim against the septal wall such that the distal rim at least partially surrounds the fossa ovalis; creating a negative pressure within the inner volume; and piercing the fossa ovalis with a piercing member to create an opening in the fossa ovalis.

In this aspect, piercing the fossa ovalis may comprise advancing a piercing member through the fossa ovalis.

In this aspect, the piercing member may include an RF electrode, wherein piercing the fossa ovalis comprises activating the RF electrode.

In this aspect, the piercing member may include a needle, wherein piercing the fossa ovalis comprises advancing a sharp tip of the needle through the fossa ovalis.

In this aspect, the method may further comprise advancing a piercing member guide distally and into contact with an inner surface of the fossa ovalis.

In this aspect, engaging the distal rim against the septal wall may include forming a seal.

In this aspect, the method may further comprise steering the piercing member toward a target puncture site of the fossa ovalis based on a visualization collected by the visualization member.

In this aspect, the visualization may be displayed in real time.

In this aspect, the method may further comprise removing the negative pressure from within the inner volume of the inflatable member after piercing the fossa ovalis.

In this aspect, inflating the inflatable member may comprise injecting fluid within an outer volume of the inflatable member until walls of the inflatable member inflate and form a stiff frame structure.

In this aspect, the method may further comprise monitoring fluid pressure within the outer volume.

In this aspect, method may further comprise controlling fluid pressure in the outer volume based on the monitored fluid pressure.

In this aspect, the fluid pressure may automatically be controlled by computer readable instructions of an external controller.

In this aspect, the method may further comprise monitoring the negative pressure within the inner volume.

In this aspect, the method may further comprise controlling the negative pressure within the inner volume based on the monitored negative pressure.

In this aspect, the negative pressure may automatically be controlled by computer readable instructions of an external controller.

In this aspect, the method may further comprise determining when puncture through the septal wall is complete, and turning off the negative pressure when puncture through the septal wall is determined to be complete.

In this aspect, method may further comprise anchoring a portion of an elongate member that is secured to the inflatable member against right atrial tissue or an ostium of the right atrium.

One aspect of the disclosure is a tissue piercing device configured to pierce a septal wall, comprising: an elongate member having a piercing member translatable therein, the piercing member having a distal end configured to pierce tissue of the septal wall; and an ultrasound imaging catheter coupled to the elongate member, the ultrasound imaging catheter having one or more ultrasound transducers configured to provide a field of view including a target punction site of the septal wall and for guiding the piercing member toward the target puncture site.

In this aspect, the tissue piercing device may further comprise a coupling member configured to couple the ultrasound imaging catheter to the elongate member, the coupling member including first channel sized and shaped to secure the elongate member therein, and a second channel sized and shaped to secure the ultrasound imaging catheter therein.

In this aspect, the coupling member may further include one or more slits that are configured to provide access to one or both of the first channel and the second channel.

In this aspect, the coupling member may further include one or more latches that are configured to lock one or both of the first channel and the second channel closed.

In this aspect, the coupling member may further include one or more inflatable locks that are configured to lock one or more of the elongate member and the ultrasound imaging catheter to the coupling member.

In this aspect, the one or more inflatable locks may be configured to expand into one or more of the first channel and the second channel.

In this aspect, the one or more inflatable locks may be configured to expand upon inflation with fluid.

In this aspect, the elongate member may include one or more deflectable sections to steer the elongate member within the heart.

In this aspect, the ultrasound imaging catheter may include one or more deflectable sections to steer the ultrasound imaging catheter within the heart.

In this aspect, the elongate member may include one or more markers viewable by the one or more ultrasound transducers, fluoroscopy, or the one or more ultrasound transducers and fluoroscopy.

In this aspect, the one or more markers may protrude from the elongate member.

In this aspect, the one or more markers may be positioned at a distal end region of the elongate member.

In this aspect, the ultrasound imaging catheter may be an intracardiac echocardiography catheter.

In this aspect, the elongate member may further include a piercing member guide, wherein the piercing member is translatable within the piercing member guide.

In this aspect, the piercing member may have a sharp distal tip.

In this aspect, the piercing member may have a distal end having a radiofrequency (RF) electrode adapted to apply RF energy to the septal wall sufficient to create an opening within the septal wall.

In this aspect, the distal end may be sufficiently sharp to mechanically pierce through the septal wall.

In this aspect, the distal end may have a blunt tip.

In this aspect, the elongate member may include an inflatable member adapted to assume an expanded configuration when inflated with fluid, wherein the inflatable member defines an internal volume when in the expanded configuration.

In this aspect, the elongate member may further include a visualization member positioned within the internal volume and to provide a second field of view through a distal end of the inflatable member.

One aspect of the disclosure is a method of piercing a septal wall, comprising: delivering an inflatable member while in a contracted configuration within the right atrium, wherein the inflatable member includes a distal membrane; supplying fluid to an outer chamber within a wall of the inflatable member until the inflatable member achieves an expanded configuration, wherein fluid pressure within the outer chamber is maintained a second fluid pressure; supplying fluid within the closed inner chamber to fill the closed inner chamber, wherein fluid pressure within the closed inner chamber is maintained at a first fluid pressure that is different than the second fluid pressure; engaging the membrane against the septal wall; and piercing the membrane and the septal wall with a piercing member to create an opening in the septal wall.

In this aspect, the method may further comprise providing a field of view through the membrane and including visualizing the fossa ovalis with a visualization member, wherein piercing the septal wall comprises piercing the fossa ovalis.

In this aspect, the first fluid pressure and the second fluid pressure may be maintained by one or more external controllers.

In this aspect, piercing the membrane and the septal wall may comprise advancing a sharp distal tip of the piercing member through the membrane and the septal wall.

In this aspect, piercing the membrane and the septal wall may comprise contacting an activated RF electrode of the piercing member with the membrane and the septal wall.

In this aspect, the second fluid pressure may be greater than the first fluid pressure.

One aspect of the disclosure is a system comprising: a tissue piercing device comprising: an inflatable member having a wall with an outer chamber, wherein the inflatable member is adapted to assume a conical shape when the outer chamber is filled with fluid, wherein the conical shape defines a closed inner chamber including a distal membrane, wherein the closed inner chamber is adapted to hold a tissue piercing member; one or more controllers operationally coupled to the tissue piercing device, wherein the one or more controllers is adapted to maintain a first fluid pressure within the closed inner chamber and to maintain a second fluid pressure within the outer chamber, the first fluid pressure different than the second fluid pressure.

In this aspect, system may further comprise a visualization member within the closed inner chamber, the visualization member arranged to provide a field of view through the membrane and including the fossa ovalis.

In this aspect, the one or more controllers may be external to the tissue piercing device.

In this aspect, piercing the membrane and the septal wall may comprise advancing a sharp distal tip of the piercing member through the membrane and the septal wall.

In this aspect, piercing the membrane and the septal wall may comprise contacting an activated RF electrode of the piercing member with the membrane and the septal wall.

In this aspect, the second fluid pressure may be greater than the first fluid pressure.

These and other aspects are described herein.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIG. 1 illustrates an exemplary tissue piercing assembly.

FIGS. 2A and 2B illustrate a sectional and side view, respectively, of an exemplary tissue piercing assembly.

FIG. 3 illustrates an exemplary field of view from an exemplary optic from an exemplary tissue piercing assembly.

FIGS. 4A-4C illustrate section views of a heart showing an exemplary method of piercing a fossa ovalis using an exemplary tissue piercing assembly.

FIGS. 5A-5C illustrate section views of a portion of a heart showing an exemplary method of piercing a septum wall using a tissue piercing assembly.

FIGS. 6A-6C illustrate various views of an exemplary tissue piercing assembly having a plurality of splines.

FIGS. 7A-7D illustrate various views of an exemplary tissue piercing assembly having a plurality of optical elements.

FIGS. 8A-8B illustrate various views of an exemplary assembly that may not include a piercing member and/or piercing member guide.

FIGS. 9A-9F illustrate various views of an exemplary secondary device that may be delivered separately from or secured to a tissue piercing assembly.

FIG. 10 illustrates an exemplary tissue piercing assembly having one or more optical elements secured to an inner surface of the inflatable member.

FIGS. 11A-11B illustrate an exemplary tissue piercing assembly having a distal open region and configured to hold negative pressure.

FIGS. 12A-12E illustrates an exemplary tissue piercing assembly that includes an intracardiac echocardiography (ICE) catheter.

FIGS. 13A-13C illustrate various views of an exemplary coupling member.

FIGS. 14A-14B illustrate various views of an exemplary coupling member that is a variation of the coupling member of FIGS. 13A-13C.

FIG. 15 illustrates an exemplary coupling member used to couple an ICE catheter with a tissue piercing device.

FIG. 16 illustrates an exemplary placement of a coupling member.

FIG. 17 illustrates an exemplary piercing member that includes a needle.

FIG. 18 illustrates an exemplary piercing member that includes an RF electrode.

FIG. 19 illustrates a block diagram of an exemplary tissue piercing system.

FIG. 20 is a flowchart illustrating an exemplary method of piercing the septal wall using a piercing device having closed inflatable member.

FIG. 21 is a flowchart illustrating an exemplary method of piercing the septal wall using a piercing device having an open inflatable member adapted to accept negative pressure.

### DETAILED DESCRIPTION

This disclosure is related to methods of piercing a FO and devices and systems adapted and configured for performing the same. The methods may be performed prior to or as a part of any number of procedures for which it is desired to create an opening in a FO, examples of which are set forth herein.

One aspect of the disclosure herein includes methods of imaging with one or more optics that provide a field of view towards the interatrial septum and/or FO during the FO piercing process. This provides better direct visualization of the tissue than existing procedures that rely on x-ray and TEE imaging, which provides higher confidence that a device is in a desired position against the FO prior to deploying a piercing device (e.g., needle or other coiled device) through tissue.

One aspect of the disclosure herein includes inflatable members that define a closed inner inflatable chamber or volume that when inflated or expanded allows one or more optics to provide the field of view towards the FO to facilitate device positioning against the FO. In some embodiments the inflatable member may include a second inflatable chamber that is considered part of the structure of the inflatable member, where the second inflatable chamber may be inflated to provide enhanced stiffness to a region of the inflatable member, which is described in more detail herein. In embodiments that includes a second inflatable chamber, the second inflatable chamber may be considered an outer inflatable chamber, with the main inflatable chamber being considered an inner (radially) inflatable chamber, additional exemplary details of which are described below.

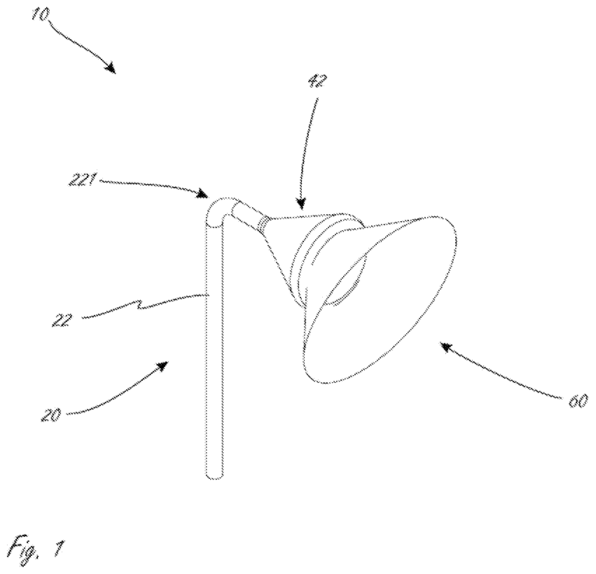

FIG. 1 illustrates an exemplary tissue piercing assembly 10 (“piercing assembly”), also referred to herein as tissue piercing device (“piercing device”). In this embodiment, piercing assembly 10 includes inflatable member 42 secured to a distal end of an elongate member 20. The inflatable member 42 may be configured to transition between a contracted configuration and an expanded configuration (e.g., as shown in FIG. 1). For example, the inflatable member 42 may be configured to remain in a contracted configuration during delivery through the patient's vessels, radially expand to the expanded configuration once at or near the septal wall, and radially contract back to the contracted configuration for transport out of the patient's body once a procedure is complete. In some embodiments when not inflated, the inflatable member 42 may easily contract but not necessarily collapse. The collapsing may follow from withdrawing the inflatable member 42 into a delivery catheter. When expanded, the inflatable member 42 can define an inner volume or chamber (also referred to herein as a first volume/chamber) that is configured to hold one or more one or more elements (e.g., optical element(s), piercing member(s) and/or piercing member guide(s)), a fluid (e.g., saline solution), and/or negative pressure. In some embodiments, a distal end of the member 42 may be configured to interface with the septal wall.

In some cases, at least a portion of the walls of the inflatable member 42 is expandable by inflation, such as by introduction of fluid therein. For example, the wall of the inflatable member 42 may include one or more outer volumes (also referred to herein as a second volume/chamber) that is/are configured to hold fluid and provide rigidity to the inflatable member 42. The walls of the inflatable member may be comprised of one or more flexible materials that can compress into the contracted configuration, and be inflated to radially expand and form a rigid frame that defines the inner volume. The walls may have a layered structure and/or include internal reinforcements to provide a desired shape and thickness to the walls once inflated. The elongate member 20 may include a catheter 22 having a lumen that is adapted to carry fluid (e.g., saline solution) distally toward the outer volume of the inflatable member 42 to expand the walls of the inflatable member 42 and/or to the inner volume of the inflatable member 42. In some examples, one or more portions of the elongate member 20 is deflectable to allow steering of the inflatable member 42.

The assembly 10 may include one or more optics (not shown) that are positioned and adapted to provide one or more fields of view 60. The one or more optics may be used to visualize a target puncture site (e.g., FO or other part of the septal wall). In some examples, the one or more optics are within, or configured to move within, the inner volume of the inflatable member 42. The shape of the inflatable member 42, when expanded, may be shaped and sized to provide a relatively large field of view 60. In some cases, the distal end of the inflatable member 42 may have a larger cross-section (e.g., diameter) than a proximal portion of the inflatable member 42. In some examples, the inflatable member 42 may have a cone shape when in the expanded configuration, as shown in FIG. 1. Any of the optics or optical elements as used herein may include imaging elements such as, without limitation, one or more cameras, one or more charged coupled devices (CCDs), one or more CMOS devices, and one or more illuminators such as optical fiber(s), LEDs, etc., including any combination thereof of any of the optics or optical elements.

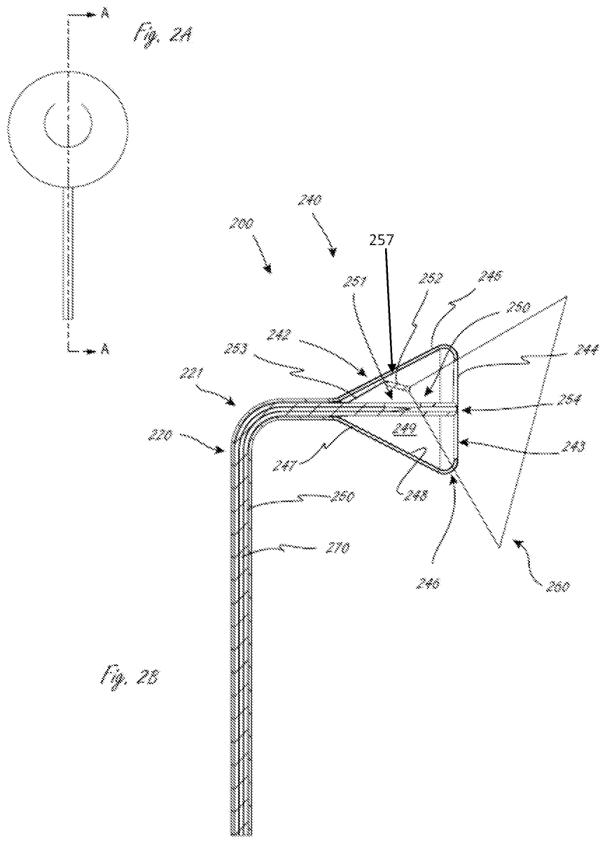

Piercing assembly 10 may include a piercing member guide that may be adapted to traverse within an inner lumen of the catheter 22 of the elongate body 20, and to be advanced to a position within the inflatable member 42. The piercing member guide may be adapted and configured to receive therein a piercing member that has a distal end configured to pierce a FO. Piercing assembly 10 may include a piercing member (e.g., needle and/or RF electrode) that is within or adapted to be advanced to a position within inflatable member 42. The piercing member may have a distal end that is configured to pierce the FO. Any of the piercing assemblies herein may include one or more of the components described with reference to FIG. 1. This disclosure makes reference to tissue piercing assemblies, and it is intended that any of the tissue piercing assemblies herein need not include in the assembly the piercing member that is configured to pierce tissue. It is thus understood that the tissue piercing assemblies and the piercing member that is configured to pierce tissue may be separate components, and that the tissue piercing assemblies may guide a separate tissue piercing member therethrough to the FO. FIG. 2A illustrates a sectional view of an exemplary piercing assembly. Piercing assembly 200 includes elongate member 220 and inflatable member 240. Inflatable member 240 in this embodiment includes proximal region 242 and distal region 243, wherein at least a portion of distal region 243 is further distally than at least a portion of proximal region 242 when inflatable member 240 is inflated. In this example, inflatable member 240 is a closed inflatable member, and distal region 243 may also be considered a closed distal region of an inflatable member in this example. In this example elongate member 220 is coupled to inflatable member 240, either directly or indirectly.

Piercing assembly 200 further comprises piercing member guide 250, a distal region of which is shown disposed within inflatable member 240. Piercing member guide 250 includes a lumen therein that ends in distal opening or port 254. The lumen and port are sized and configured to receive therethrough a piercing member 270, which may have sharpened distal end as shown in this example. Piercing member guide 250 may be axially movable relative to elongate member 220, and piercing member 270 may be axially movable relative to elongate member 220 and to guide 250.

Piercing assembly 200 includes optical element 252 (e.g., a camera and/or ultrasound imaging element (e.g., ICE)), which is configured and adapted to provide field of view 260 when activated. In this example, optical element 252 is coupled to arm 253, which may be coupled to a shaft, such as a shaft that is part of piercing member guide 250 or elongate member 220. In this exemplary embodiment, arm 253 may be biased to revert to the deployed configuration as shown, which is radially outward from the shaft of guide 250, which allows optic 252 to provide the field of view 260 that is oriented towards and the distal region 243 and the FO, and which includes at least a portion of distal region 243. The shaft of guide 250 includes depression, recessed region, or pocket 251, which is configured to receive optic 252 therein, which can allow the optic to be stored therein during delivery, which may decrease the overall delivery profile of the assembly during delivery. Arm 253 may be made of an elastic material such as nitinol, for example without limitation. Upon inflation of inflatable member 240, arm 253 may deploy to its deployed configuration as shown (such as by self-expansion or self-deployment), providing field of view 260. FIG. 2 illustrates closed inflatable member 240 in an expanded and inflated configuration. In some examples, at least a portion of the elongate member 220 (e.g., shaft of guide 250) and/or the arm 253 may be steerable (e.g., deflectable) to align the optic 252 according to a desired field of view. In some examples, the optic 252 is positioned in a fixed location within the inflatable member 240.

FIG. 2B illustrates an exemplary expanded and inflated configuration of inflatable member 240. In this example, inflatable member 240 includes a distal region 243 that includes a pierceable distal membrane 244. In this example, membrane 244 in distal region 243 has a flat or generally flat configuration, as shown. The flattened configuration may make it easier to pierce the membrane with a piercing device compared to a distal region that is non-planar such as a portion of a sphere (e.g., spherical cap or dome) or another rounded configuration (e.g., toroidal, ellipsoid, etc.). The flattened configuration may also make it easier to conform to the shape of the FO when the membrane is urged against the FO. The flattened configuration may make it easier for the membrane and FO to be tensioned together during piercing. Inflatable member 240 in this embodiment has a conical configuration, as shown, with distal region 243 generally forming the base of the cone or conical configuration, with proximal region 242 generally forming at least a portion of the tapering surface(s) of the cone or conical configuration.

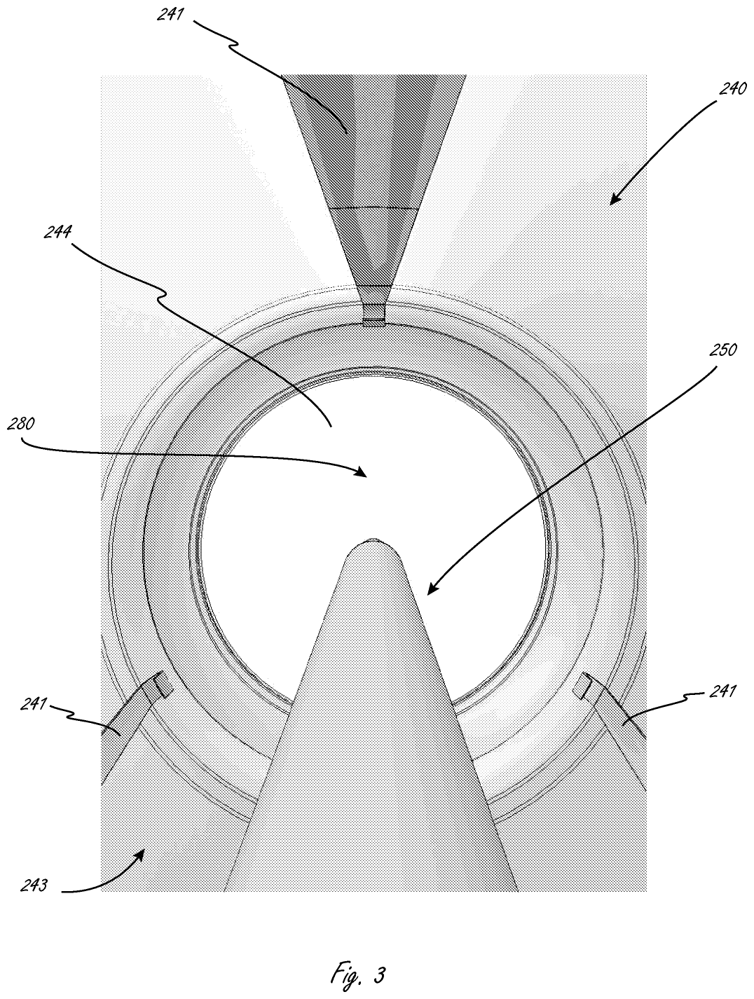

When the inflatable member is in an atrium, it is exposed to blood. The field of view through the distal region 243, such as through membrane 244. will include blood that may be contacting and adjacent to the distal region 243 of the inflatable member. To be able to visualize the septum and FO with the optic(s), the inflatable member is preferably pushed up against and into contact with the septal wall and FO. Doing so will generally displace at least some of the blood that was between the membrane 244 and the wall. Theoretically, the distal region of the inflatable member may be pushed against septal wall tissue to essentially stop the flow of blood between the distal region and the wall, although there may be some modest amount of residual blood. Once the membrane 244 is pushed against the tissue, the optic(s) can visualize the FO as the field of view may be oriented towards the wall and FO. The membrane 244 may be sufficiently transparent (e.g., optically transparent) to allow visualization therethrough by optic(s) (e.g., optical element 252). FIG. 3 illustrates an exemplary field of view from an exemplary optic, which includes, in the context of the piercing assembly shown in FIG. 2, distal surface 244, inflatable member 240 generally, proximal region 243, and guide member 250. Other exemplary and optional components are shown, which are described herein in the context of other figures, such as plurality of splines 241. Since the distal surface 244 of the inflatable member may be transparent or largely transparent, FO 280 can be seen through the membrane 244 when the optic(s) (e.g., one or more cameras and one or more illuminators) are activated.

The distal surface 244 may be a wide variety of material that is both pierceable and at least partially transparent, such as, for example without limitation, a silicone elastomeric material, and/or polyethylene terephthalate (PET).

In some embodiments the inflatable member may include a first region that is stiffer than a second region of the inflatable member. In some exemplary embodiments, a proximal region of the inflatable member (e.g., proximal region 242 in FIG. 2B) may be stiffer than a distal region of the inflatable member (e.g., distal region 243). A stiffer proximal region of the inflatable member may help urge and maintain the inflatable member against the septal tissue to minimize blood flow between the wall and the inflatable member, and a relatively less stiff distal region (e.g., distal membrane) may be easier to pierce with a piercing member 270. The flattened configuration of the membrane may also help conform to the shape of the FO, which can help clear blood from between the membrane and the FO. The relative stiffnesses between the distal and proximal regions may be achieved in a variety of ways. For example only, a proximal region may comprise relatively stiffer polymeric material than a distal region. Additionally, for example only, a proximal region (e.g., inflatable walls) may have a thickness that is greater than a distal region (e.g., membrane). In some examples, a proximal region (e.g., region 242 in FIG. 2B) may comprise a sealed inflatable region 245 that includes at least first and second layers that define a second chamber therein, the second chamber being adapted to be inflated to increase fluid pressure therein, which can be used to increase the stiffness of the proximal region. In some examples, the fluid pressure (e.g., threshold pressure) within the outer (second) volume/chamber may be maintained in a range from about 250 mmHg to about 5000 mmHg. FIG. 2B illustrates an inflatable region 245 that includes an outer layer 247 and an inner layer 248, wherein the outer layer 247 and the inner layer 248 may form walls of a frame structure of the inflatable member 240. The inner and outer layers 247 and 248 in this example define the outer (second) chamber that is configured to hold a fluid. This stiffness of the frame structure of the inflatable member 240 may be adjusted by controlling the fluid pressure within the outer (second) chamber. In some cases, the pressure may be increased and/or decreased using one or more fluid actuators (e.g., pressure servomotors), as described herein.

Inflatable region 245 may be considered to include an outer inflatable chamber, wherein inflatable member 240 generally defines an inner (first) inflatable chamber or volume 249 defined by layer 248. That is, guide 250 and optic 252 are considered within the inner inflatable chamber 249.

In some embodiments, the walls of the device 200 are not inflatable yet define an inner volume 249. The inner volume 249 may itself be inflatable (e.g., include a distal membrane), or may not be inflatable (e.g., include a distal opening). In cases where the d inner volume 249 includes a distal opening, the inner volume 249 may be configured to hold a negative pressure when the device 200 is sealed against the septal wall, as described herein.

In some embodiments, the distal end of piercing member guide 250 is not coupled to the inflatable member 240, but is it disposed or is adapted to be disposed therewithin, as shown in FIG. 2B. In some alternative embodiments, a piercing member guide may be coupled to the inflatable member.

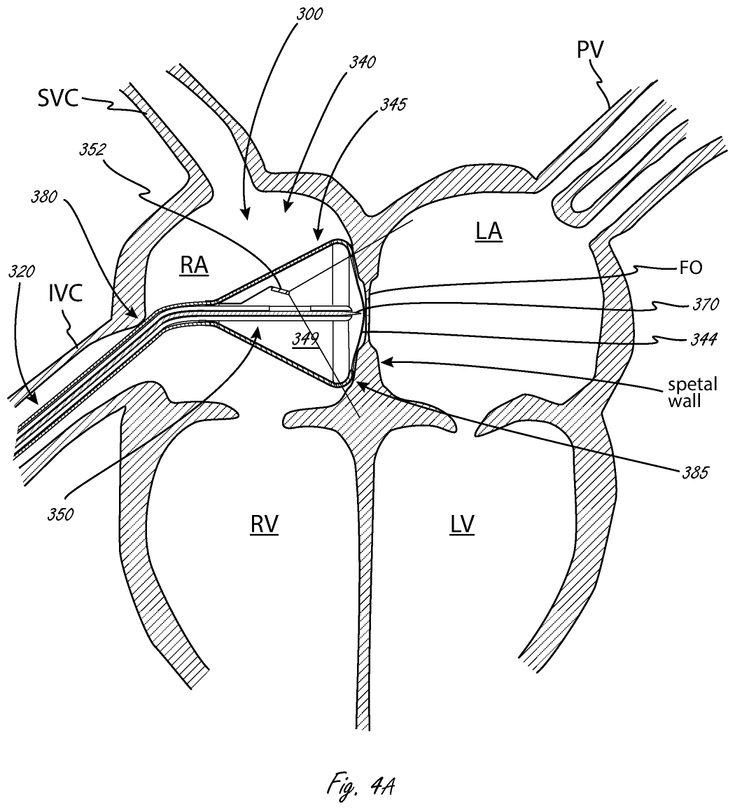

FIGS. 4A-4C illustrate exemplary steps that may be included in a method of piercing a FO. FIG. 4 shows an exemplary inflatable member 340 of an exemplary tissue piercing assembly 300 disposed in a right atrium “RA” in an inflated configuration. A full access method is not shown, but for delivery through the IVC in this example, the inflatable member 340 is delivered in a smaller delivery non-fully inflated configuration.

After being advanced to the RA, the inflatable member may be inflated by delivering an inflation fluid from one or more fluid sources through an inflation lumen in elongate member 320 and into the inner chamber or volume 349 that is defined by inner surfaces of inflatable member 340. In this example, inflatable member also includes an inflatable region 345, which is described elsewhere herein. Either before, during, or after fluid is delivered into chamber 349, fluid may also be delivered into the chamber of inflatable region 345. The same or different fluid may be used to inflate chamber 349 and the chamber of inflatable region 345. The fluid delivered to the different chambers may come from the same fluid reservoir or different fluid reservoirs. In some examples, the fluid used to inflate multiple (e.g., two) chambers may be delivered down a common lumen (e.g., single fluid conduit) within or attached to at least a portion of elongate member elongate member 320. In some examples, the elongate member 320 include separate lumens (e.g., two or more fluid conduits) within or attached to at least a portion of the elongate member 320 for supplying fluid to multiple (e.g., two) chambers. The multiple lumen configuration may be conducive to independently controlling pressures within the multiple chambers. For example, the inner volume 349 may be maintained at a first pressure, and the outer volume (within the walls of the inflatable region 345) may be maintained at a second pressure different than the first pressure. In some examples, an inner volume (e.g., 349) may be maintained around the mean arterial pressure within the atrium+100 mmHg or −700 mmHg. This may be significantly lower than the pressure within the outer volume within the walls of the inflatable region 345. This is because higher pressure within the walls may be required to maintain a desired stiffness of the walls and for the inflatable member 340 to maintain a desired frame shape. In addition, it may be desirable for a membrane 344 at a distal end of the inflatable member 340 to be sufficiently inflated to provide a field of view therethrough, yet be flexible enough to conform to the septal wall, including the FO. In some embodiments, the multiple inflatable chambers are considered not to be in direct fluid communication. In some examples, they may receive fluid from different fluid sources. In other examples, they may receive fluid from a common fluid source. In some embodiments, however, there may be one or more fluid apertures in an inner surface of the inflatable member 340 that allows fluid to pass from chamber 349 and into the second outer chamber of inflatable region 345, in which case the two chambers are considered to be in direct fluid communication.

Fluid has been delivered into at least one of the inflatable chambers in FIG. 4A to at least partially inflate the inflatable member. Additionally, inflatable member 340 has been urged at least partially into contact with the atrial side of the septal wall at one or more locations, such as at location 385, which may be an annular contact region. Optic 352 can be activated and provides, and may be maneuvered (e.g., steered) to provide, a field of view that includes the FO. The inflatable member is urged against the wall to help displace blood between the inflatable and the interatrial wall. Some initial deformation (e.g., bowing) of the distal membrane 344 may occur as shown after some initial inflation due at least partially to the increase in pressure within chamber 349, and optionally also to the relatively less stiff distal surface/membrane 344. In some embodiments there may not be much deformation (e.g., bowing), if any at all.

FIG. 4B illustrates an exemplary step in which the fluid pressure continues to be increased within inflatable member 340, such as by continued delivery of fluid into the chamber 349 (see FIG. 4A). This may cause additional deformation (e.g., bowing) of the distal membrane, as shown, which may also cause additional contact between the distal membrane and the atrial wall, as shown. This may help further reduce the amount of blood between the atrial wall and the inflatable member, which may improve visualization of the FO to ensure proper placement of the inflatable member and/or the piercing member guide 350. In FIG. 4B, it can be determined by viewing the image (e.g., on an external monitor in communication with the one or more optical elements) that the distal face 344 and/or FO are distended outward and/or that the inflatable member is in an appropriate position to deploy the piercing member 370. By being able to directly visualize the FO, more confidence is gained that the piercing member 370 will be advanced through the FO.

FIG. 4C illustrates the piercing member 370 after it has been advanced and pierced through both the distal membrane 344 and the FO, and extends into the left atrium “LA,” creating an opening in the FO. Any number of additional steps and procedures can be performed utilizing the opening in the FO.

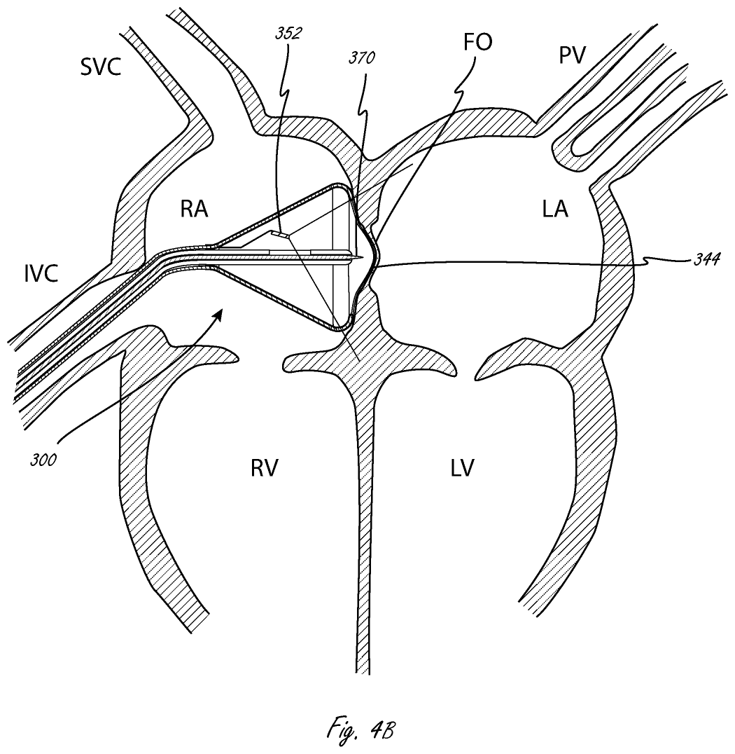

FIGS. 5A-5C illustrate an exemplary sequence of method steps that may be performed during a procedure that creates an opening in an interatrial septum, any of which may be combined with any other suitable method sequence to step(s) herein. The tissue piercing assembly shown in FIGS. 5A-5C may be any suitable tissue piercing assembly herein. FIG. 5A illustrates an inflated inflatable member that is also in contact with an atrial septal wall, with a distal surface partially bowing distally due to fluid pressure. As set forth herein, the configuration of the distal membrane may help at least partially conform the membrane to septal wall tissue, which may help displace blood between the membrane and the septal wall. Visualization can help confirm proper placement. FIG. 5B illustrates piercing member guide 350 being advanced distally relative to the position shown in FIG. 5A, pushing into contact against the distal membrane 344, which is pushed in contact against the FO. This step may be performed instead of or in combination with an increase in fluid pressure to cause additional contact between the inflatable member and septal wall tissue. Again, visualization with the optical element(s) can help confirm and/or reconfirm that the guide member placement is desired. FIG. 5C illustrates the piercing member 370 after being advanced (e.g., translated distally within the piercing member guide 350) and piercing through the distal membrane 344 and the FO.

In any of the embodiments herein, pressure at one or more locations in and/or on the inflatable member may be monitored. Monitoring pressure may be used as part of a feedback mechanism to monitor and/or control one or more properties of the inflatable member, such as pressure within the main chamber and/or a secondary inflatable chamber. For example without limitation, pressure within inflatable chamber 349 (see FIG. 4A) may be monitored as part of a method that is adapted to dynamically maintain a pressure within inflatable chamber 349 above a certain threshold or within a certain range (for example) to help, for example only, make sure membrane 244 is maintained in contact against the FO. In addition to or alternatively, pressure within any of the secondary inflatable chambers herein may be monitored as part of a method that is adapted to dynamically maintain a pressure within the secondary inflatable chamber above a certain threshold or within a certain range (e.g., within the blood pressure range of the right atrium), for example. Dynamic control of pressure may incorporate one or more fluid source, and one or more fluid control mechanisms that are adapted to control flow fluid, such as a pump. For example, first and second inflatable chambers may be in fluid communication with a single fluid source, but first and second fluid control features (e.g., pumps, valves) may facilitate individual control of flow delivery to each of the different inflatable chambers. Alternatively, any of the different inflatable chambers may be in fluid communication with different fluid sources.

Monitoring pressure may be performed during any portion of the piercing procedure, including before the FO is pierced, during, or after the FO is pierced. Monitoring may be performed periodically or continuously, which may help alert of a sudden or unexpected drop in pressure within the chamber.

Monitoring may be integrated into an automatic and/or manual method that includes monitoring pressure and manually or automatically (e.g., via a computer executable method/algorithm) controlling pressure in response thereto. Controlling pressure may include delivering additional fluid into the inflatable chamber to, for example, increase and/or maintain fluid pressure in the inflatable chamber. The computer executable method/algorithm may be executed by and/or stored on one or more controllers (e.g., one or more computers). At least a portion of the controller(s) may be adapted to be external to the patient's body (e.g., at a proximal end of the tissue piercing assembly). In some examples, the controller(s) is/are configured to independently control the fluid pressure within different chamber/volumes of the inflatable member. For example, the controller(s) may be configured to maintain pressure within an outer (second) chamber at a first fluid pressure (or first range of pressures), and to maintain pressure within an inner (first) chamber at a second fluid pressure (or second range of pressures) that is different than the first fluid pressure (or first range of pressures). In other examples, the controller(s) is/are configured to maintain fluid pressure within different chamber/volumes of the inflatable member at (e.g., substantially) the same fluid pressure (or same fluid pressure range).

One or more pressure sensors may be secured to the inflatable member, wherein the one or more sensors may be adapted to sense or be indicative of fluid pressure (e.g., resistive, capacitive, and piezoelectric pressure sensors) at one or more locations of the inflatable member. For example without limitation, one or more pressure sensors may be coupled to an inner portion of the inflatable member, such as an inner surface of the inflatable member that defines chamber 249. For example without limitation, any number of sensors may be coupled to any one of splines 241 or other stiffening element to help secure the sensor to the inflatable member. Additionally, any number of electrical wires that may be coupled to any of the sensors herein may be coupled to a spline or other stiffening element, the wires extending along the stiffening element and extending proximally to a location outside of the elongate member and/or within a secondary inflatable chamber. Any number of sensors herein may also be coupled to any of the guide members herein, such as guide 250 shown in FIG. 3. Any of the sensors herein may alternatively be adapted to be fluidly coupled to the inflatable medical device and may reside in the user interface portion of the system disposed outside the patient.

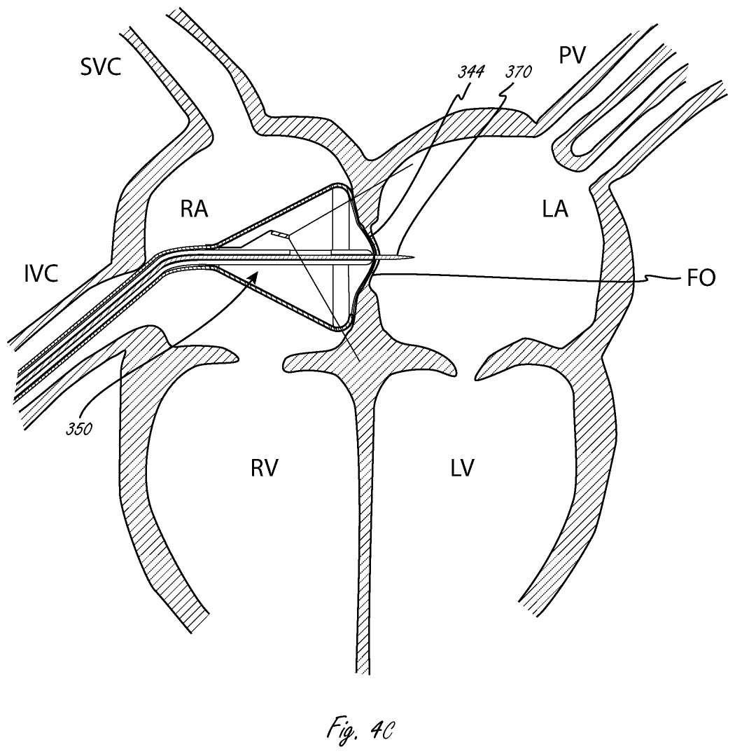

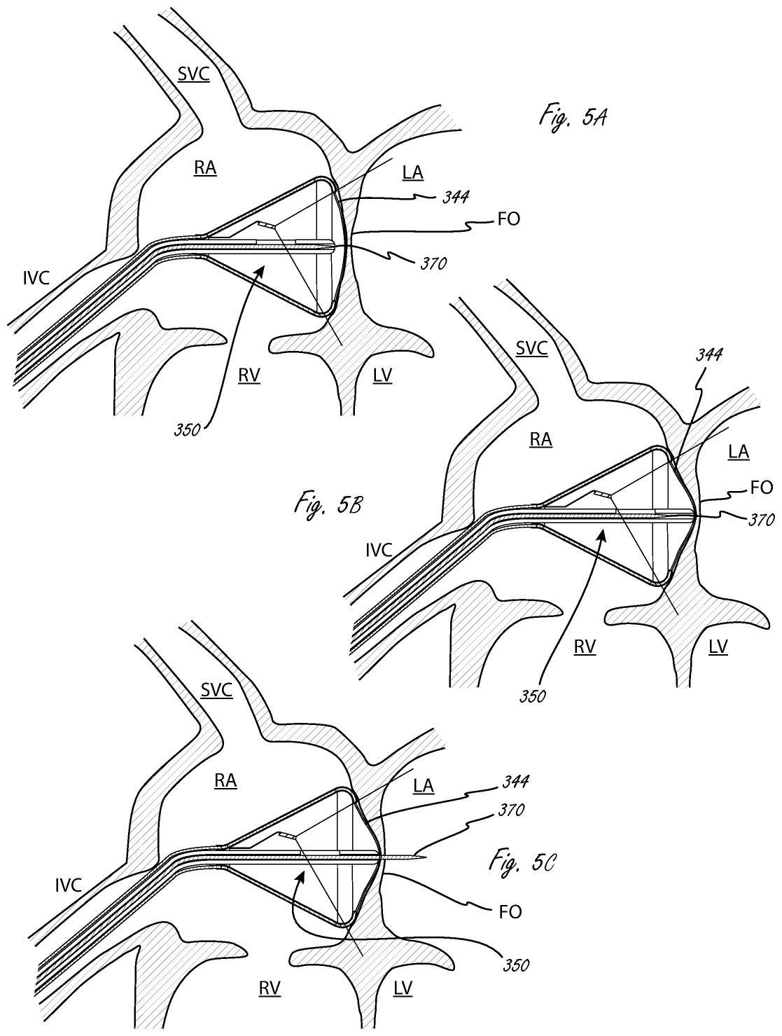

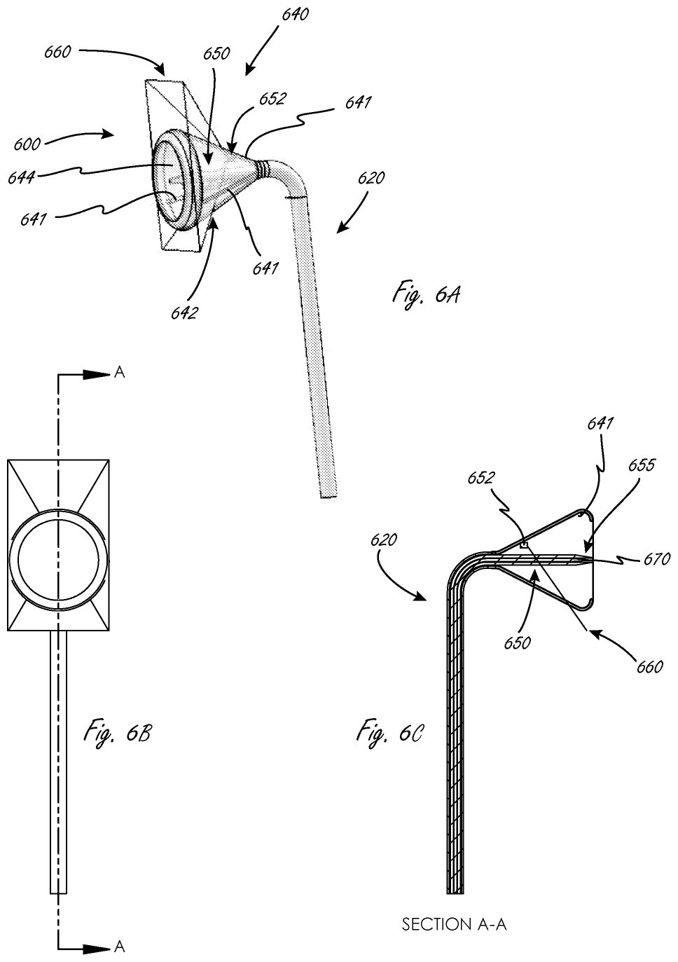

FIG. 6A illustrates an exemplary tissue piercing assembly 600 with inflatable member 640 in an inflated configuration. Inflatable member 640 may include any component and/or functionality of any other adaptable inflatable member herein. An exemplary difference between inflatable member 640 herein and that shown in FIGS. 2A and 2B is that inflatable member 640 includes a plurality of splines 641 that are secured to at least the proximal region of inflatable member 640. The splines may be made from a variety of materials, such as an elastic material like Nitinol, for example without limitation.

In any of the embodiments herein, the tissue piercing assembly may include one or more splines, such as one spline, two splines, three splines, four splines, five splines, or more, such as ten splines. In any of the embodiments herein the tissue piercing assembly may include from one to twenty splines, for example. Use of the term spline herein is not meant to limit the term spline to any particular structure, configuration, or adaptation herein.

In some embodiments, any one of a plurality of splines 641 may have an unstressed configuration in which they are disposed radially inward directly adjacent and around, and optionally into contact with, piercing member guide 650. In some embodiments that include splines, the one or more splines may not be in direct contact with a piercing member guide, but may be near in proximity thereto. In some embodiments an optic (e.g., camera) may be stored within or in contact with some aspect of the guide member (e.g., see FIG. 2B), the position of which may prevent one or more splines from directly contacting the guide member when the one or more splines are in an unstressed configuration. In some embodiments, however, one or more of the position, size, or storage state of an optic may allow for one or more splines to have unstressed states in which they directly contact a guiding member. For example, an optic and electronics may be disposed on one side of a guiding member, and a spline may be situated on an opposite side of the guiding member, such as 180 degrees around the guiding member, and in contact with a guiding member.

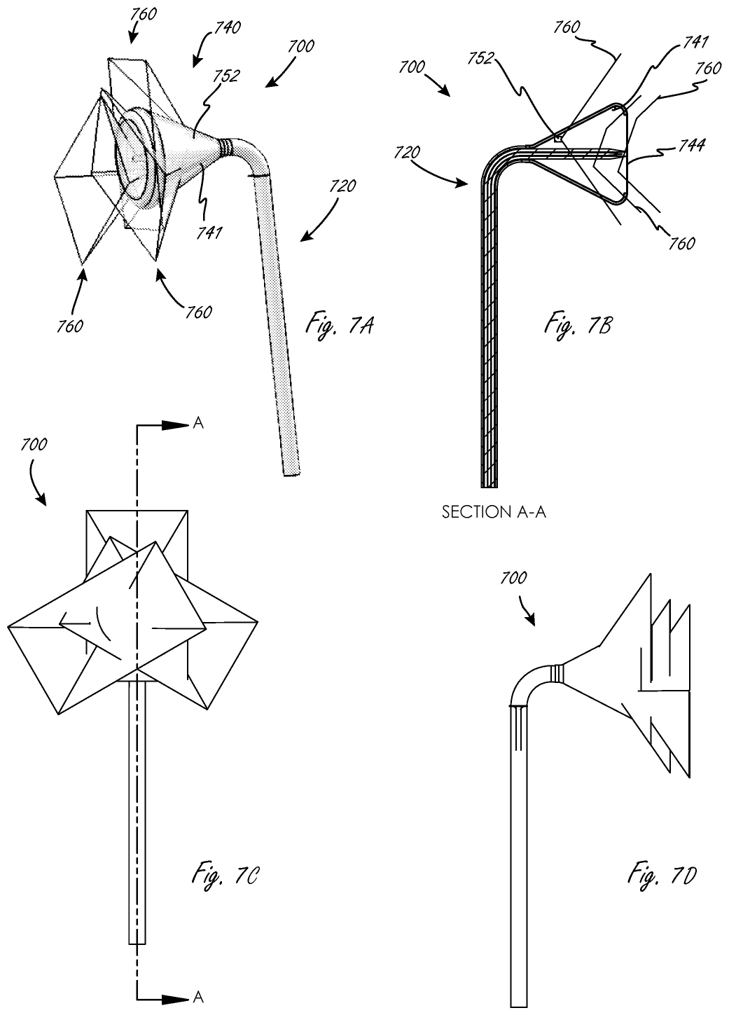

In some embodiments, which may be used with the examples in FIG. 3 and FIGS. 7A-7D for example, the guide may not be advanced distally into the inflatable member until the splines are deployed outside. In these examples, the one or more splines may help provide stiffness to the inflatable member during its delivery to the target location.

Inflation of the inflatable member causes the chamber to inflate, which causes splines 641 to be moved radially outward to the expanded configuration as shown. The splines are stressed in their expanded configurations, and are adapted to revert towards their collapsed configurations when the fluid pressure in the inflatable member is not large enough to keep them in an expanded configuration. When the assembly is to be removed from the patient, the inflation fluid can be pulled from the chamber through elongate member 620, or simply allowed to passively exit the chamber through irrigation ports in the inflatable member. The splines can thus assist in collapsing the inflatable member 640 for removal from the patient. In some alternative embodiments, splines 641 (or any splines herein) may be configured to automatically revert to the expanded configuration as shown, and may be collapsed by an outer sheath or shaft that is moved axially relative to the inflatable member. Field of view 660 is shown. Features of assembly 600 that are the same as or similar to other features herein in other assemblies may be labeled with similar reference numbers even if not expressly described. Exemplary optical element 652 is shown, which may be any optical element herein. Any other suitable feature of any other tissue piercing assembly herein may be incorporated with assembly 600, and any suitable feature of assembly 600 (e.g., splines) may be incorporated into any other tissue piercing assembly herein.

FIG. 6B shows a front view of the assembly 600 and FIG. 6C shows section A-A shown in FIG. 6B, which includes one of the plurality of splines 641. FIG. 6C also illustrates an exemplary optional characteristic of piercing member guide and dilator 650, which has a sharped distal end 655, which may allow the guide to function as a dilator that is advanced through the opening created when piercing member 670 is pierced through the inflatable member and the FO. Guide 650 is axially movable relative to inflatable member 640. Any of the tissue piercing assemblies herein may include a piercing member guide and dilator with a sharped distal end, such as is shown in FIG. 6C.

FIGS. 7A-7D illustrate views of an exemplary tissue piercing assembly that may include any suitable feature of any other tissue piercing assembly herein. Additionally, FIGS. 7A-7D illustrate an exemplary tissue piercing assembly with a plurality of optical elements 752 (e.g., plurality of cameras and/or illuminators such as LEDs). In this exemplary embodiment, any optical element and/or associated wires may be secured to any of the one or more splines 741, examples of which are described herein. An optical element 752 is shown secured to exemplary spline 741 in the cross-sectional view of FIG. 7B. While not shown in FIG. 7B, this exemplary tissue piercing assembly includes first, second, and third optical elements, each secured to one of three splines. In this example, the optical elements are secured to respective splines such that the optics are axially spaced apart from each other, which is discernible from the fields of view 760 shown in FIGS. 7A and 7D. Axially spacing the optics in this manner may help maintain a smaller collapsed profile of the inflatable member, compared to profile in which the optics are all located at the same axial location. This may also help create an overall larger field of view for visualization compared to a single optical element, which can be seen in the overlapping fields of view for the plurality of optics shown in FIG. 7C. In this example, the optics are equidistantly spaced circumferentially around the guide, and in this example the three optics are spaced 120 degrees. In other embodiments, a different number of optics may create different angular spacing, such as having 2 optics 180 degree apart, or four optics 90 degrees apart. The circumferential spacing (e.g., equidistant) may also help maintain a smaller collapsed profile of the inflatable member.

As is described elsewhere herein, in any of the embodiments herein the splines may be biased to collapsed, closed configuration (closer to parallel with a long axis of the guide than when in a deployed configuration). In these examples, inflation of the inflatable member may cause the one or more optics to be moved radially outward with the inflatable member, providing the field of views shown in FIGS. 7A-7D. Electronic connections (e.g., wire(s)) attached to any of the optic(s) herein may also be secured to the respective splines and/or inflatable polymeric material, and may extend proximally through elongate member 720 to one or more external devices (e.g., one or more computers) that may facilitate creating and providing the image on a remote display (e.g., monitor).

In any of the embodiments herein in which the tissue piercing assembly includes a second, outer, inflatable chamber, any of the splines may be contained within the wall of the second inflatable chamber.

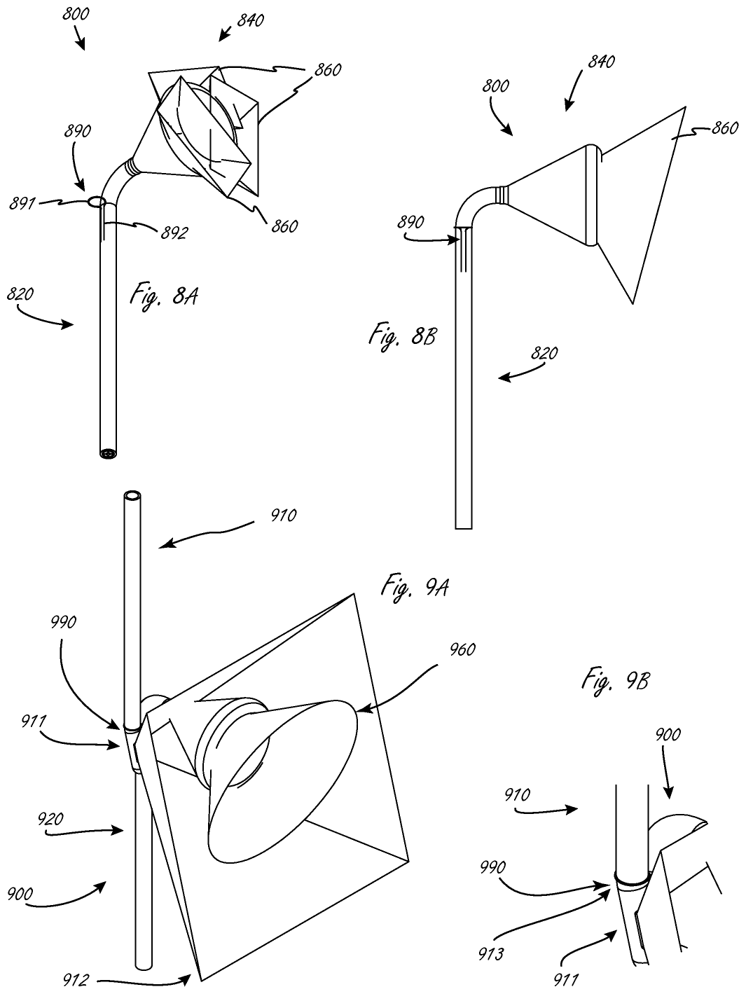

FIGS. 8A and 8B illustrate an exemplary embodiment of an assembly 800 that may be used to pierce tissue but is not necessarily limited to such uses and may not include features or components that adapt it for piercing tissue (e.g., it may not include a piercing member or piercing member guide). Assembly 800 may include any individual feature, component, or functionality of any tissue piercing assembly herein, including any combination thereof. Assembly 800 includes elongate member 820 and inflatable member 840, which in this example includes a plurality of optics, which provide for the plurality of fields of view as shown. Assembly 800 also includes coupling member 890, which is adapted and configured to couple with a secondary medical device that can be delivered separately from assembly 800 but coupled or secured thereto. The coupling member 890 may facilitate coupling to a secondary medical device and increasing the stability between assembly 800 and the secondary medical device, examples of which are described below. In this exemplary embodiment, coupling member 890 includes a coupling element 891 that includes at least one surface configurated to interface with and at least partially stabilize an elongate medical tool, and in this example has an annular configuration that is configured to receive therein a medical tool with a cylindrical shaft, as shown. Coupling element 891 may be secured to elongate member 820 with a wide variety of securing techniques, with as via a securing element 892 that forms an extension of coupling element 891.

In any of the embodiments herein, the tissue piercing assembly may include more than one optic (e.g., more than one camera), each of which has a field of view. In any of these embodiments, any of the optics may have a field of view that includes only a portion of the distal membrane (rather than the entirety of the membrane), and adjacent fields of view may have some degree of overlap (e.g., see FIG. 7C), such that a composite field of field may be created (e.g., with imaging software) by stitching the different fields of view together. The stitched composite image may include a complete image of the membrane. Allowing for composite imagery to create the desired field of view may provide more options with respect to optic component spacing, such as how and where to integrate and position one or more optics in an inflatable member. Any of these considerations may help create a smaller delivery profile by optimizing the placement of components by minimizing their positional overlap for delivery.

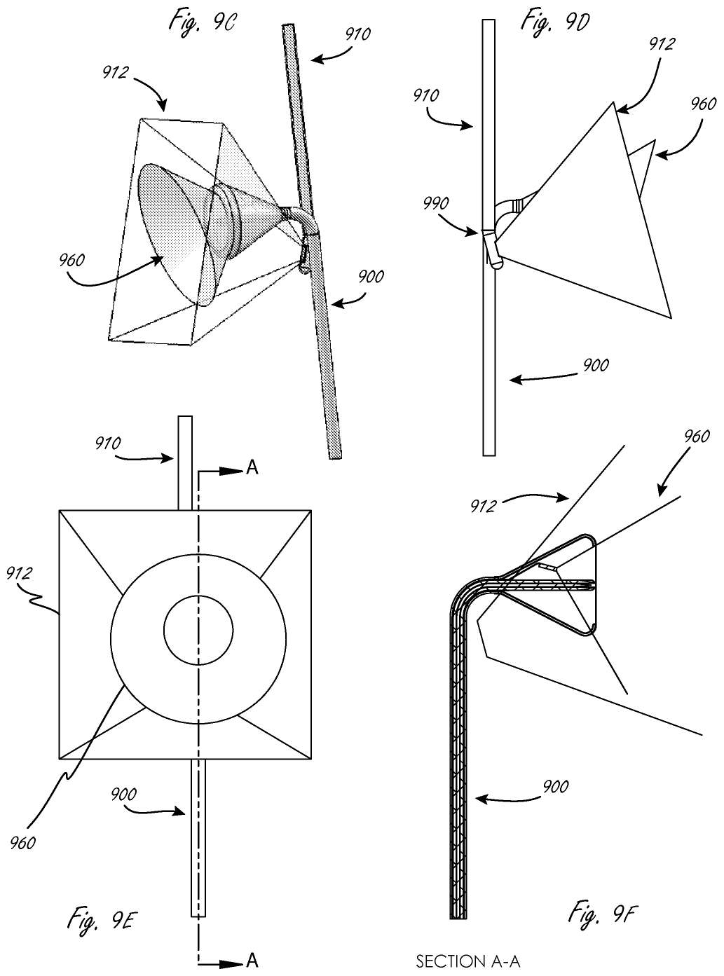

FIGS. 9A-9F illustrate an example of secondary device 910 that can be delivered separately from assembly 900, but is shown after being secured thereto. In this example, secondary medical device 910 is coupled to coupling member 990 of assembly 900, which increases the stability between assembly 900 and secondary medical device 910. As an example only, in this embodiment secondary device 910 may be a visualization device, such as an ultrasound imaging device, and includes visualization member 911, which may comprise an ultrasound imaging transducer. Exemplary secondary device imaging field of view 912 is shown in FIG. 9A, as is the field of view 960 of an optic that is part of assembly 900 (which may be any of the one or more optical elements or optics herein). Coupling member 990 may facilitate relative stability between assembly 900 and secondary imaging tool 910, which can provide additional visualization of one or more anatomical locations during the medical procedure. FIG. 9B highlights the coupling or capture 913 of secondary tool 910 via the coupling member 990.

In this exemplary embodiment, coupling member 900 comprises an annular or looped region that is sized and configured to receive secondary tool 910 therethrough, such as a generally cylindrically shaped shaft thereof. Coupling member 990 may have an annular configured to help stabilize an elongate shaft of the secondary tool 910. It is understood that coupling member may have a variety of other configurations. Additionally, other forces may be used to coupling an assembly to a secondary medical device. For example, magnetic forces may be used to help facilitate securing the assembly to the secondary device. Magnetic materials on the assembly and secondary tool, for example, may facilitate magnetic coupling. In other embodiments, the coupling may be assisted with a tether on the secondary device or the assembly that hooks onto a corresponding feature of the other device.

In any of the embodiments herein, the tissue piercing assembly may include an obturator including one or more electrodes (e.g., RF electrodes), and the secondary tool may be an imaging tool (e.g., intracardiac echocardiography (ICE) catheter) that may provide visualization for a medical procedure using the RF electrode(s) performed on the left atrial side of the septum, such a mapping and/or RF ablation procedure.

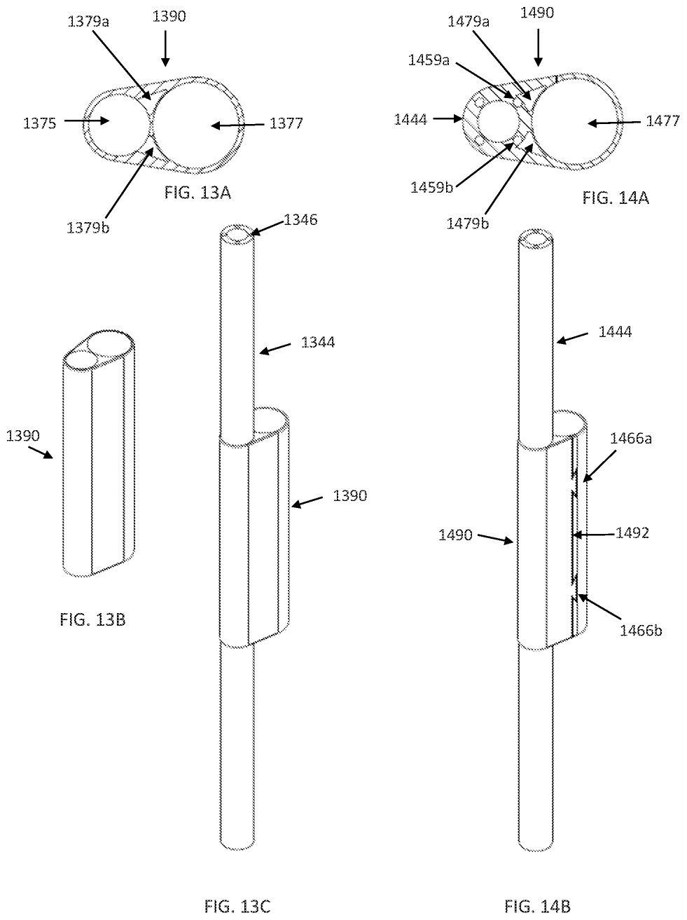

FIGS. 13A-13C illustrate various views of another exemplary coupling member 1390. In this example, the coupling member 1390 includes a first channel 1375 and a second channel 1377. The first channel 1375 may be shaped and sized to accept a first elongate member 1344 (e.g., first catheter) for a first device (e.g., a piercing device), and the second channel 1377 may be shaped and sized to accept a second elongate member (e.g., second catheter) for a second device (e.g., an imaging device). The sizes and shapes of the first channel 1375 and the second channel 1377 may vary depending on the sizes and shapes of the corresponding first and second elongate members. The coupling member 1390 may be adapted to allow the first or second device to be delivered into the body separately. For example, an imaging device (e.g., ICE) catheter may be delivered to the heart first to collect images of the septal wall. Then, first elongate member 1344 with a piercing device may be advanced to the heart by sliding the coupling member 1390 along the imaging device catheter residing in channel 1377. The coupling member 1390 may also increase overall stiffness of the combined first and second elongate members, thereby increasing the stability of the combined structure.

In this example, the coupling member 1390 includes inflatable locks 1379a and 1379b adapted to controllably engage with and prevent movement of the first and second elongate members. For example, inflatable locks 1379a and 1379b may each include inflatable membranes that have access to the first channel 1375 and the second channel 1377. Upon inflation with fluid, the inflatable locks 1379a and 1379b may expand into the first channel 1375 and the second channel 1377 to engage with the corresponding first and second elongate members, thereby preventing axial movement thereof. When the inflatable locks 1379a and 1379b are deflated, the first and second elongate members may be free to traverse axially within the corresponding first channel 1375 and second channel 1377. The fluid for the inflatable locks 1379a and 1379b may supplied by one or more fluid channels that are, for example, external to the coupling member 1390 and that are in fluid communication with the inflatable locks 1379a and 1379b. In some cases, the first elongate member 1344 includes openings 1346 for accommodating pull-wires to steer (e.g., deflect) one or more sections of the first elongate member 1344.

It is noted that the coupling member 1390, and any coupling member described herein, may be configured to couple elongate members (catheters) of any type of device(s). For example, the first or second device may be any type of visualization device (e.g., camera, CCD, CMOS and/or ultrasound (e.g., ICE)), illumination device (e.g., LED and/or optical fiber), tissue engagement device (e.g., tissue-piercing device and/or tissue-anchoring device), guidewire, and/or prosthetic implant.

FIGS. 14A-14B illustrate various views of an exemplary coupling member 1490 that is a variation of the coupling member 1390. In this variation, the first elongate member 1444 is incorporated into, or fixedly coupled to, the coupling member 1490. The second channel 1477 includes a slit 1492 that is configured to open and provide access to the second channel 1477. In some cases, the slit 1492 is hingeably opened. The coupling member 1490 includes latches 1466a and 1466b that are configured to lock the second channel 1477 closed. Inflatable locks 1479a and 1479b are configured to expand upon inflation into the second channel 1477, and engage with/lock the second elongate member in place.

In any of the embodiments herein, the coupling member may have a first state or configuration configured to allow the secondary tool to be placed therein, and a capture state or configuration that is configured to capture the secondary tool. The first configuration may be larger in size to facilitate placement of the secondary tool therein, and the second configuration may be smaller to help capture or secure the secondary medical tool. In some embodiments the coupling member may be configured to lasso the secondary tool to capture the secondary tool.

FIGS. 9C and 9D illustrates additional views of assembly 900 coupled to secondary device 910, including their respective fields of view. FIG. 9D shows a front view of the assembly 900 coupled to secondary medical device 910, including their respective fields of view. FIG. 9F shows section A-A from FIG. 9E, including exemplary features of assembly 900. FIGS. 9E and 9F illustrate how long axes of shafts of the assembly and secondary tool are offset when the two tools are secured together.

In some examples, a secondary medical device may be advanced along a different access pathway or route than the assembly. For example, without limitation, any of the assemblies herein (e.g., tissue piercing assembly) may be advanced through an IVC and into a right atrium. A secondary medical device (e.g., device 910) may be advanced down through the SVC and coupled to the assembly via the coupling member, examples of which are described herein. The coupling members herein used in this context may also be referred to as docking members or docking stations of the assembly.

The secondary device may have a wide variety of functionality and need not be an imaging tool. For example only, the secondary device may include any therapeutic functionality (and/or other diagnostic function), such as an ablation device or used to deliver an implantable.

Imaging capabilities of one or both of the assembly and secondary tool may be used to facilitate their coupling.

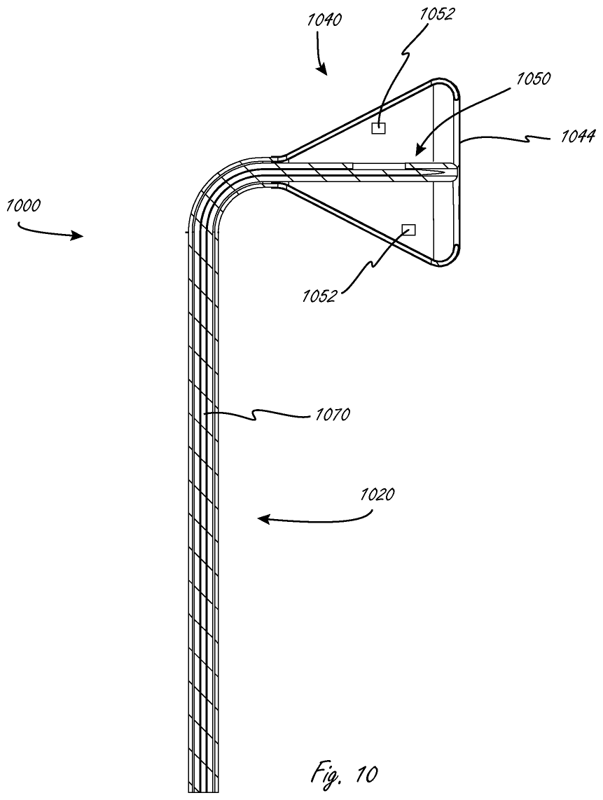

FIG. 10 illustrates an exemplary tissue piercing assembly 1000 that may include or incorporate any other feature or function of any suitable tissue piercing assembly herein (e.g., second inflation chamber of the inflatable member). In this example, one or more optical elements 1052 (e.g., camera(s)) may be secured to an inner surface of the inflatable member, such as a polymeric surface. Electronics attached to the optical element(s) are also secured to the inner surface of the inflatable member. Inflation of the inflatable chamber may cause the optics to move radially outward, facilitating their preferred fields of view of the distal surface 1044 of the inflatable member.

In any of the embodiments therein that includes a secondary inflatable chamber (e.g., FIG. 10), any of the optic and associated electronics (e.g., wires, optical fibers, cameras, illuminators such as LEDS, etc.) may be disposed within the secondary inflatable chamber alternatively or in addition to being secured to a radially outer or radially inner surface thereof. In any of these examples, any spline or other stiffening element may also be disposed at least partially within the secondary inflatable chamber or secured to an inner or outer surface thereof.

Within a secondary inflatable chamber in this context refers to be disposed between a radially outermost surface and a radially inner most surface of the secondary inflatable chamber. In these embodiments, an inner surface of the second inflatable chamber may be at least partially transparent to facilitate fields of view of any optic(s) disposed therein, such as fiber optics or cameras.

Any of the elongate members herein (e.g., 220, 320, 420, 620, 920, 720, may comprise one or more shafts, exemplary constructions of which are known. For example, elongate member 220 in FIG. 2 may comprise a shaft that includes one or more polymeric material, optionally one or more reinforcing members such as braided materials. Any of the piercing member guides may also comprise one or more shafts. Any of the inflatable members herein may be coupled to a shaft of any of the elongate member shaft herein, such as is shown in FIG. 2.

Any of the piercing member guides herein may be axially movable relative to any of the elongate members herein and/or the inflatable members. For example, guide 250 may be axially moved relative to elongate member 220 and inflatable member 240. Any of the assemblies herein may be coupled to handles adapted to be gripped by a physician, which may include one or more components that facilitate axially movement of any of the piercing member guides and/or piercing members herein. Handle mechanism concepts and construction that may be used to facilitate this relative axial movement are generally known, such as one or handle actuators (e.g., rotatable knobs, axially moveable sliders, etc.), any of which may be incorporated into a handle that is part of any of the tissue piercing assemblies herein.

Any of the piercing members herein (e.g., needles) have a sharpened distal end that is configured to pierce through the distal membrane of the inflatable member as well as the FO.

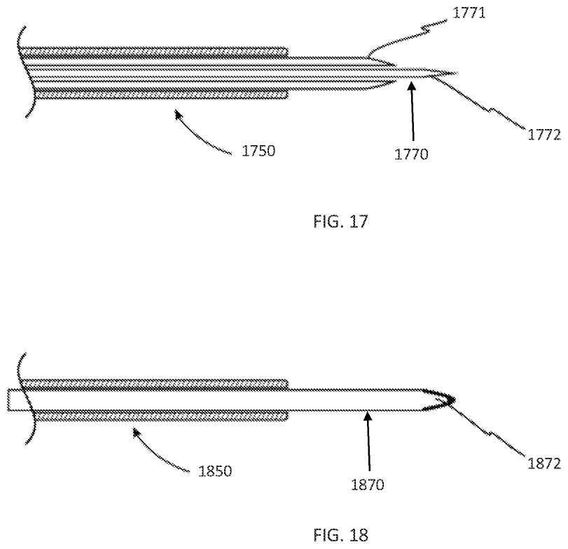

In any of the embodiments herein, the tissue piercing member may include a needle. FIG. 17 illustrates an exemplary piercing member 1770 that includes a needle having a sharp distal tip 1772 for piercing through tissue (e.g., FO). In this example, the piercing member 1770 is positioned within a lumen of a piercing member guide 1750 to guide placement of the piercing member 1770. A dilator 1170 may be used to control translational movement of the piercing member 1770 within the piercing member guide 1750.

In any of the embodiments herein, the tissue piercing member may incorporate a radiofrequency (RF) element/electrode. FIG. 18 illustrates an exemplary piercing member 1870 having a distal tip that includes a radio frequency (RF) electrode 1872. The RF electrode 1872 may be adapted to apply RF energy to the tissue to facilitate creating an opening in the FO (e.g., via tissue ablation). The RF element may be operationally coupled to an RF generator that is controllable directly by a user and/or one of more computers. In any of these embodiments, the tissue piercing member 1870 may or may not have a distal end that is shaped to mechanically pierce tissue. For example, the tissue piercing member 1870 may not have a sharpened distal end or a blunt distal end.

In any of the embodiments herein, the tissue piercing member may be (or be part of) a guidewire that is subsequently used to guide delivery of one or more medical devices or tools into the heart (e.g., into the left atrium).

In any of the embodiments herein, the piercing member may be insulated except for its distal end, and therefore electrical impedance may be used to (e.g., part of a computer executable method/algorithm) differentiate and indicate the position of the distal end of the piercing member, such as if the piercing member is in the inflatable member, in contact with the septum, or in the left atria. Any of the inflatable members herein may have a distal region that includes a distal membrane that is pierceable by the piercing member. Distal membranes may be relatively thin material, such as a flexible polymeric material. Any of the inflatable members herein may include a region, such as inflatable region 245 in FIG. 2B, that is separately inflatable from a central internal chamber, such as inflatable region 245 in FIG. 2B.

In any of the embodiments herein the membrane may also include a slit valve.

One of more assembly components herein may be adapted to be steerable, any of which may facilitate proper positioning of one or more assembly components. One or more shafts may include known steering components and functionality, such as one or more pull-wires incorporated in one or more shafts. In any of the embodiments herein, the assembly may have a steerable section that is proximal to the inflatable member, such as section 221 as shown in FIG. 2B. In these examples, the steering may be incorporated into one or both of the elongate member (e.g., 220) and the piercing member guide (e.g., 250). For example, an elongate member may have a pull-wire integrated therein to facilitate steering, which may be one-way steering in section 221. When delivered through an SVC and into a right atrium, for example, the assembly will be steered in a particular direction towards the interatrial septum, so one-way steering may be all that is required in that section. In some examples, the one or more shafts (e.g., elongate member (e.g., 220) and/or piercing member guide (e.g., 250)) may include multiple steerable sections (e.g., 2, 3, 4, 5 or more steerable sections) along an axial length the shaft(s).

In any of the assemblies herein, the piercing member guide (e.g., 250) may have a distal steerable section that is inside of or adapted to be moved inside of the inflatable member. For example, with reference to FIG. 2B, reference number 250 also points to an exemplary steerable section in the guide. A steerable section in the distal region of the guide facilitates steering the guide, which may help ensure the piercing member is advanced into the desired anatomical location. Steering the guide may be particularly helpful when used while visualization the distal region of the inflatable member, with an exemplary field of view shown in FIG. 3, in which guide 250 may incorporate steering in the section shown. Additionally, it may be helpful to position the assembly generally, and fine-tune the piercing member trajectory by steering the guide while visualizing the distal region of the inflatable member and the septal wall to help ensure the piercing member is advanced through the FO.

A section of any of the elongate members herein may also be steered to help create an assembly configuration that helps anchor or increase the stability of the inflatable member within a right atrium. This is shown generally in FIGS. 4A-5C, in which FIG. 4A shows elongate member 320 engaging tissue 380 near the ostium between the right atrium and inferior vena cava at location. Engagement with the tissue 380 can create a counter-traction in a direction opposite of the distal end of the piercing member device 300, thereby securing the distal end of the piercing member device 300 against the septal wall. That is, engagement with the tissue 380 near the ostium can act as an anchoring point for the piercing member device 300. The distance or length between the bent region of the elongate member (when steered) and the end of the inflatable member (if present) can cause the inflatable member to be stabilized or anchored in the right atrium. If that distance is too short it may not anchor, and if that length is too long it may not be possible to situate the inflatable member properly to deploy the piercing member through the FO. It is of note that anchoring is not an essential aspect of this disclosure, but it may help stabilize the assembly.

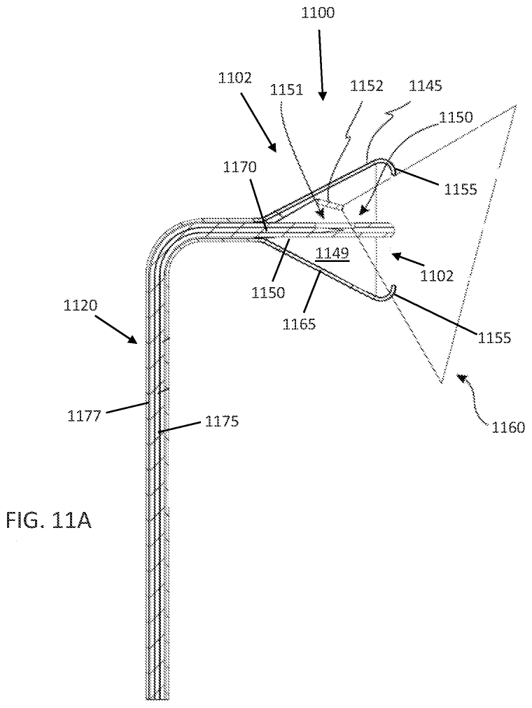

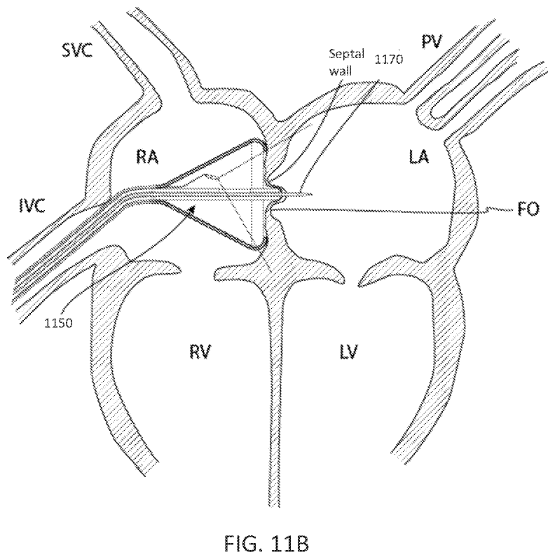

FIGS. 11A and 11B illustrate an exemplary piercing assembly 1100 that may include any of the features of the piercing assemblies of FIGS. 2A-2B, 3, 4A-4C, 5A-5C, 6A-6C, 7A-7D, 8A-8B, 9A-9F and 10, except that the inflatable member 1145 has a distal end having an opening 1102 (e.g., does not include a distal membrane 244). Similar to other embodiments described herein, the inflatable member 1145 may be layered to form a sealed outer volume/chamber adapted to hold fluid for expanding and stiffening the inflatable member 1145. The distal end of the inflatable member 1145 includes an edge or rim 1155 that surrounds the opening 1102. The distal rim 1155 may include an engagement surface adapted to engage with the septum wall to provide anchoring points for the inflatable member 1145. The rim 1155 may surround an area of the septum wall that at least partially includes the target puncture site (e.g., FO). The rim 1155 may have a shape in accordance with a shape of the inflatable member 1145. For example, a cone-shape shaped inflatable member 1145 may form a ring-shaped (annular) rim 1155. The rim 1155 may be adapted to contact the septal wall and create a seal, when engaged with the septal wall, that is sufficient for the inner volume/chamber 1149 of the inflatable member 1145 to hold a negative pressure. In some examples, the rim 1155 may have an atraumatic surface (e.g., a rounded/curved surface) to minimize tissue damage and/or to improve sealing with the septal tissue. As described herein, a negative pressure may correspond to a pressure lower than an ambient pressure in the right atrium. The negative pressure may draw the septal wall (e.g., FO) into the inner volume/chamber 1149. In some cases, septal wall is drawn against the piercing member guide 1150 and/or the piercing member 1170 to facilitate piercing of the septal wall.

A proximal end of the elongate member 1120 may be adapted to operationally couple with one or more suction devices (e.g., vacuum pump(s)). The elongate member 1120 may be adapted to supply the negative pressure to the inner volume/chamber 1149 and to supply fluid to the outer volume/chamber 1165. In some examples, the elongate member 1120 includes a first conduit or channel 1175 (e.g., negative pressure conduit) in communication with the inner volume/chamber 1149 (e.g., within the cone), and a second conduit or channel 1177 (e.g., fluid conduit) in communication with the outer volume/chamber 1165 (e.g., within the wall of the cone).

The inflatable member 1145 is adapted to be sufficiently rigid (in the expanded configuration) to withstand collapsing when the negative pressure is applied. The negative pressure within the inner volume/chamber 1149 can draw in the septal wall (e.g., including the FO) against the piercing member guide 1150, thereby providing a traction force on the FO against which the piercing member 1170 may be advanced. Once the piercing member 1170 has punctured the septal wall (e.g., FO), the negative pressure can be removed from the inner volume/chamber 1149. This may prevent or reduce the blood drainage from the left atrium. Once the piercing procedure is complete, the inflatable member 1145 may be disengaged from the septal wall and deflated to its contracted state for removal from the patient's body. In an alternate embodiment, not shown, the negative pressure may be applied with the piercing member 1170 extended into the FO, wherein the application of the negative pressure may pull the FO over the piercing member 1170, thus facilitating the puncture of the FO.

In some embodiments, the negative pressure within the inner volume/chamber 1149 is maintained within a maximum of about −700 gauge. In some embodiments, negative pressure within the inner volume/chamber 1149 is maintained within a maximum of about 60 mmHg.

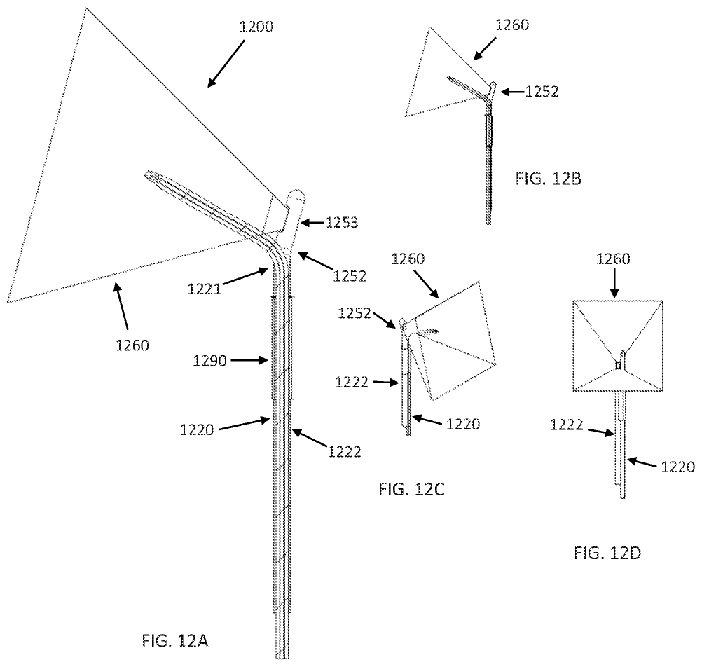

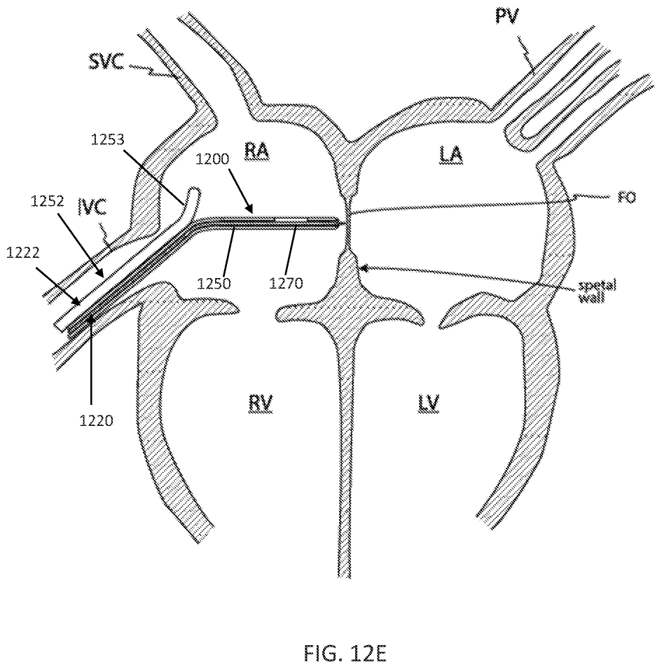

FIGS. 12A-12D illustrate an exemplary tissue piercing device 1200 used in conjunction with an ultrasound imaging device 1252 (e.g., ICE catheter). The ultrasound imaging device 1252 can include an ultrasound imaging transducer 1253 at a distal region of an elongate member 1222. The imaging transducer 1253 is adapted to provide a field of view 1260 for imaging through blood. The elongate member 1222 of the ultrasound imaging device 1252 may be coupled to the elongate member 1220 of the tissue piercing device 1200 using a coupling member 1290. The coupling member 1290 may be one of the coupling members 890, 990, 1390 or 1490. The distal region of the elongate member 1222 may be steerable (e.g., deflectable), for example, by one or more pull-wires incorporated in the elongate member 1222. When in used, the elongate 1222 may be steered to direct the imaging transducer 1253 toward and provide a field of view 1260 including the target puncture site (e.g., FO). As described herein, the elongate member 1220 of the tissue piercing device 1200 may also be steerable (e.g., deflectable), for example, by one or more pull-wires incorporated in the elongate member 1220. This arrangement may allow the piercing member of the tissue piercing device 1200 be steered independently of the ultrasound imaging device 1252. Since the imaging transducer 1253 may image through blood, the tissue piercing device 1200 may not need an inflatable member (e.g., to shield one or more optical elements therein from blood). However, in some cases, the ultrasound imaging device 1252 may be combined with a tissue piercing device having an inflatable member with one or more imaging element (e.g., optical element(s) and/or a second ICE transducer) therein.

FIG. 12E illustrates an example use of the tissue piercing device 1200 with the ultrasound imaging device 1252 coupled thereto (coupling member not shown) within the heart. The ultrasound imaging device 1252 can be steered (e.g., deflected) to provide an image of the FO along the septal wall. The piercing member guide 1250 of the tissue piercing device 1200 may be steered (e.g., deflected) to align the piercing member 1270 toward the septal wall (e.g., FO). The piercing member 1270 is translated through the piercing member guide 1250 to puncture the septal wall (e.g., FO).

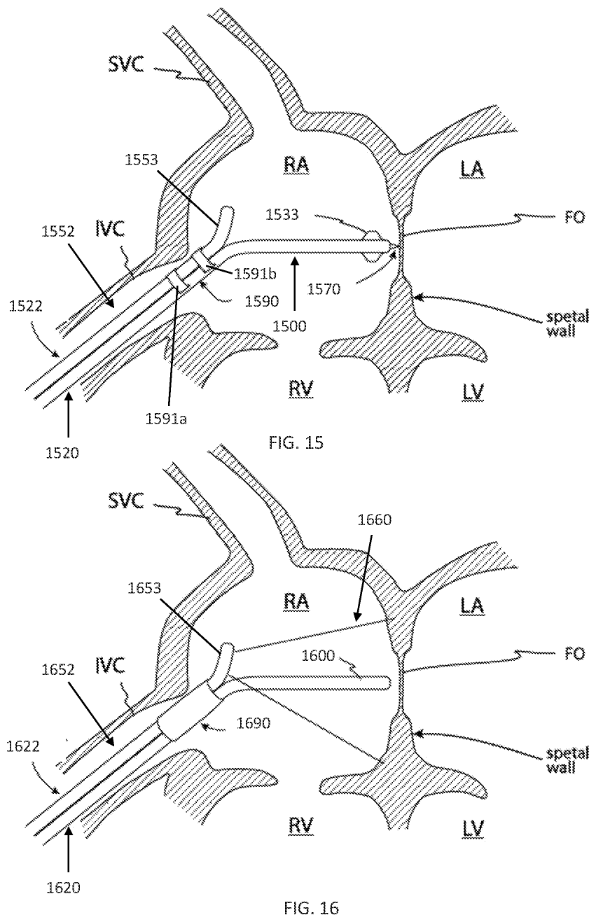

FIG. 15 illustrates an exemplary coupling member 1590 used to couple an ICE catheter 1552 with a tissue piercing device 1500. In this example, the coupling member 1590 is part of, or extends from, the elongate member 1520 of the tissue piercing device 1500 and wraps around the elongate member 1522 of the ICE catheter 1552. In this case, the coupling member 1590 includes two bands 1591a and 1591b that wrap around the elongate member 1522 of the ICE catheter 1552, although the coupling member 1590 may include any number of bands (e.g., 1, 2, 3, 4, 5, or 6 bands). One or more of the bands 1591a and 1591b may include any of the couple member features describe herein, such as one or more inflatable locks and/or one or more latches.

In any of the embodiments described herein, the tissue piercing device 1500 may include a marker 1533. In this example, the marker 1533 is at a distal region of the tissue piercing device 1500. However, the marker 1533 may be along any region of the tissue piercing device 1500. In some examples, the marker 1533 may be radio-opaque to allow visualization of the marker 1533, e.g., via fluoroscopy. This can provide additional visualization of the tissue piercing device 1500 to help guide the piercing member 1570 toward the septal wall (e.g., FO). Additionally or alternatively, the marker 1533 may extend and protrude from an outer surface of the tissue piercing device 1500 (e.g., from to tissue piercing member guide) to allow visualization of the marker 1533 via the transducer 1553 of the ICE catheter 1552 and/or an imaging device on the tissue piercing device 1500.

FIG. 16 illustrates an exemplary placement of a coupling member 1690. In this example, the coupling member 1690 is configured to couple to a region of the elongate member 1620 of the tissue piercing device 1600 and the elongate member 1622 of the ICE catheter 1622 that is proximal to steerable (e.g., deflectable) sections of the elongate members 1620 and 1622. This can allow steering (e.g., independent steering) of the transducer 1453 (to align a field of view 1660) and of the tissue piercing device 1600 (to guide placement of the piercing member).

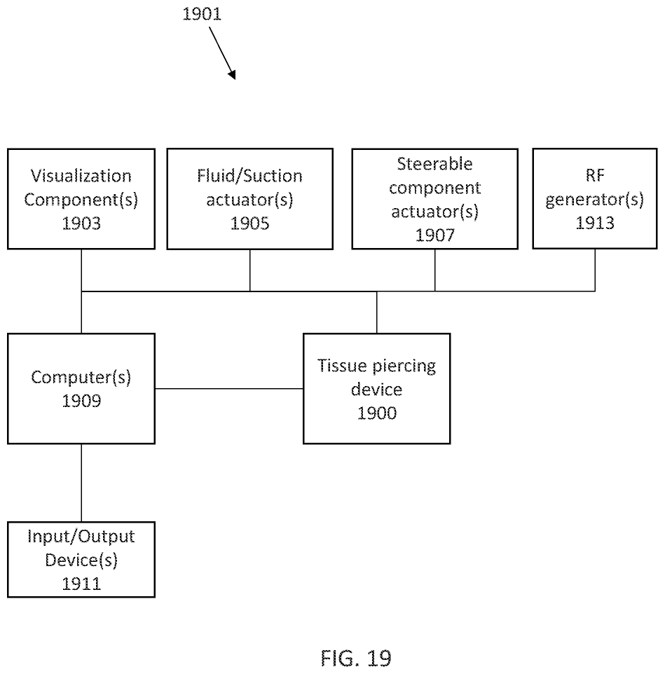

FIG. 19 illustrates an exemplary system 1901 including a tissue piercing device 1300. The system 1901 includes a one or more computers 1909 that includes memory having computer-readable instructions for performing one or more methods. The computer(s) 1309 also includes one or more processors coupled to the memory to execute the computer computer-readable instructions. The computer(s) 1909 may include an external controller for controlling one or more aspects of the tissue piercing device 1900. The computer(s) 1909 may be configured to directly or indirectly control one or more aspects related to the tissue piercing device 1900. The computer(s) 1909 may be operationally coupled to one or more visualization components 1903, one or more fluid flow and/or suction actuators 1905, one or more steerable component actuators 1907, and/or one or more radio frequency (RF) generator, which may activate one or more features of the tissue piercing device 1900. The visualization component(s) 1903 may include one or more optical fibers and/or wires that communicate with one or more visualization elements (e.g., optics and/or ultrasound (e.g., ICE) transducer) of the tissue piercing device 1900. The computer(s) 1909 may be configured to receive and store visual data collected from the optical element(s), and send data to one or more displays for displaying one or more images (e.g., video). In some examples, the computer(s) 1909 is configured to send real-time image data for real-time images collected by the optical element(s). The fluid flow and/or suction actuator(s) 1905 may include one or more fluid actuators (e.g., servomotors) for providing fluid to one or more volumes/chambers (e.g., first and/or second volumes/chambers) of the tissue piercing device 1900. The steerable component actuator(s) 1907 may include one or more steering mechanisms, such as a pull-wire mechanism, to control bending of the elongate member and/or optical element(s). The RF component 1913 may be configured to generate an RF signal delivered to one or more RF elements (e.g., tissue piercing electrode(s)) of the tissue piercing device 1900.

The computer(s) 1909 may be configured to received signals from and/or send signals to (e.g., control signals) one or more of the visualization component(s) 1903, fluid flow and/or suction actuator(s) 1905, steerable component actuator(s) 1907, RF generators, and tissue piercing device 1900. For example, the computer(s) 1909 may be configured to receive input from a user via one or more input/output devices 1911 to deflect one or more steerable components of the tissue piercing device 1900, and send signals to the tissue piercing device 1900 based on such input. As another example, the computer(s) 1909 may be configured to receive measured fluid pressure and/or negative pressure within the first and/or second volumes/chambers of the tissue piercing device 1900, and send controls signals (e.g., automatically) to the fluid flow and/or suction actuator(s) 1905 to increase and/or decrease fluid flow and/or suction to a obtain a predetermined (e.g., threshold) pressure. In some examples, the computer(s) 1909 is configured to automatically control the fluid pressure(s). As a further example, the computer(s) 1909 may be configured to send signals (either directly or indirectly) to the tissue piercing device 1900 to activate/deactivate an RF element of the tissue piercing device 1900. In some examples, the computer(s) 1909 is adapted to control one or more of the visualization component(s) 1903, fluid flow and/or suction actuator(s) 1905, steerable component actuator(s) 1907, and tissue piercing device 1900 via feedback control. The computer(s) 1909 may be configured to receive and/or send data to/from one or more input and/or output devices 1911. For example, computer(s) 1909 may be configured to receive input from one or more user interfaces (e.g., keyboard, mouse, and/or touchscreen), and/or send output to one or more devices (e.g., display and/or printer). In some examples, the computer(s) 1909 is configured to receive and/or send data wirelessly. In some examples, the computer(s) 1909 is configured to receive and/or send data via a network (e.g., internet and/or intranet).

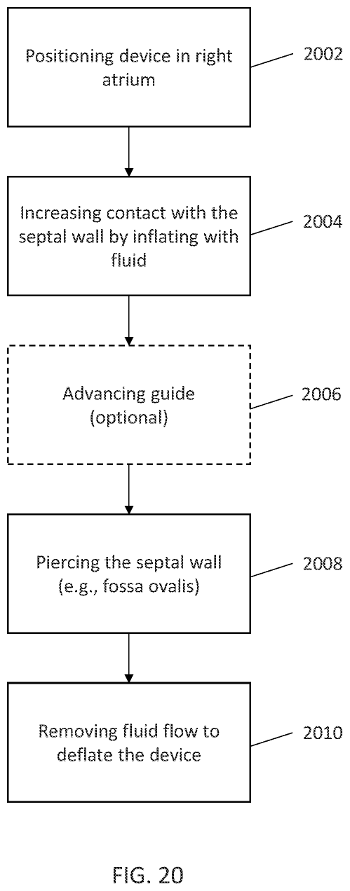

FIG. 20 illustrates an exemplary method for using a tissue piercing device having a closed distal end. At 2002, the device is positioned within the patient's right atrium and at the septal wall, where the walls of the device are inflated. Delivery of the device may involve advancing the inflatable member while in a contracted configuration via a delivery catheter through the patient's blood vessels and into the right atrium. Once in the right atrium, the inflatable member may be released from the delivery catheter and expanded into the expanded configuration, for example, by introducing fluid into the first volume/chamber and/or second volume/chamber of the expandable member. At 2004, fluid (e.g., increased fluid pressure) may be introduced into the first volume/chamber such that the distal membrane of the inflatable member can deform to increase contact with the atrial wall (e.g., FO). Once or more optical element(s) may be used to visualize the target puncture site through the distal membrane. This information can be used to steer the tissue piercing member and/or tissue piercing member guide toward the target puncture site. At 2006, a tissue piercing member guide is optionally advanced toward the target puncture site. At 2008, the septal wall (e.g., FO) and the membrane is pierced using the tissue piercing member (e.g., needle or guidewire). Once the tissue piercing procedure is complete, at 2010, the flow of fluid to the inflatable member may be stopped to deflate the inflatable member into the contracted state. The inflatable member may then be retracted into the catheter and removed from the patient's body.

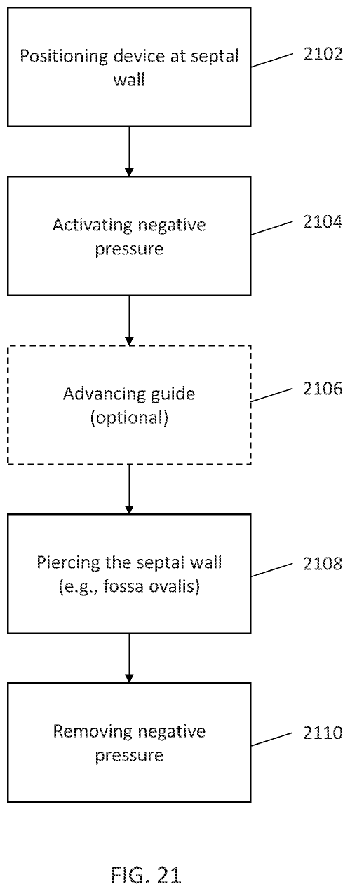

FIG. 21 illustrates an exemplary method for using a tissue piercing device having an open distal end. At 2102, the device is positioned within the patient's right atrium and at the septal wall. This may involve releasing an inflatable member from a delivery catheter and introducing a fluid into a second volume/chamber of the inflatable member, e.g., within the walls of the inflatable member, to expand the inflatable member. Upon expansion, the inflatable member can form a frame structure (e.g., conically shaped) that defines a first volume/chamber. Positioning the inflatable member may include contacting a distal rim of the inflatable member against the septal wall to form a seal. In some examples, the distal rim of the inflatable member is shaped and sized to surround at least a portion of the FO. At 2104, suction may be applied within the first volume/chamber to create a negative pressure within the first volume/chamber. The suction force may secure the inflatable member against the septal wall. The walls of the inflatable member may be sufficiently stiff to prevent collapse of the inflatable member when the suction is applied. In some cases, the suction force may pull the atrial wall proximally within the inflatable member. At 2106, a tissue piercing member guide (e.g., with the tissue piercing member therein) is optionally advanced distally toward the target puncture site. In some cases, the tissue piercing member guide is positioned in contact against the against the atrial wall (e.g., FO). At 2108, the septal wall (e.g., FO) and the membrane is pierced using the tissue piercing member (e.g., needle or guidewire). Once the tissue piercing procedure is complete, at 2110, the suction force may be stopped to remove the negative pressure from the first volume/chamber. The inflatable member may then be deflated into the contracted state, e.g., by stopping fluid flow into and removing from the second volume/chamber. The inflatable member may then be retracted into the catheter and removed from the patient's body.

Any of the steering functionality may be incorporated into a handle assembly using known techniques. For example, any number of pull-wires that are used to steer herein may be in operable communication with one or more handle steering actuators, such as rotatable knobs or axially moveable elements.

As set forth herein, visualization may help ensure the guide is in the proper position before the piercing member is advanced. The visualization via the optical element(s) may also be used to monitor that the distal surface (e.g., membrane) of the inflatable member is in contact with tissue. If the inflatable member is not in contact with tissue, blood will be seen or visualized in the field of view. This may indicate that the inflatable member needs to be advanced distally, that the inflatable member needs to be inflated more to pressure the chamber and cause the distal membrane to bow outward, and/or that the guide needs to be advanced further distally to push the distal membrane against tissue. Any combination of these actions may be performed to create more contact between the tissue and inflatable member prior to advancing the piercing member.

In any of the embodiments herein, an internal pressure of the one or more internal volumes/chambers (e.g., first and/or second internal volume/chamber) of the inflatable member may be monitored for one or more uses. For example, an internal pressure may be monitored, which may be part of a method (e.g., software, algorithm) that is adapted to perform one or more of monitoring, controlling, or changing the internal pressure. For example, monitoring internal pressure may be used to maintain a pressure within the inflatable member, or make sure it stays in a range, or that it doesn't drop below a threshold or go above a threshold, including any combination thereof. Internal pressure(s) data may be collected by one or more pressure gauges within the inflatable member and/or external to the inflatable member but in communication with the first and/or second internal volume/chamber. The internal pressure may be a positive pressure or a negative pressure. Pressure may be controlled by modifying the delivery of fluid into the inflatable chamber, such as starting, increasing, decreasing, or stopping the flow of fluid in or out of the chamber. Other aspects of monitoring and/or control of pressure may be found elsewhere herein, and maybe incorporated with any of these concepts. In any of the embodiments herein, the inflatable member may include one or more small irrigation ports in the inflatable member, including in one or both of the proximal or distal regions. Fluid may pass through the ports and into the body, which may help prevent blood from entering into the inflatable chamber. Any of the methods herein may include monitoring pressure as a way of determining how much blood may be entering into the inflatable member after the piercing member pierces the distal membrane. Any of the methods herein may include monitoring pressure and increase it if needed to prevent blood from entering the inflatable member. Any of the methods herein may include monitoring pressure to prevent leakage from the inflatable member. Any of the methods herein may include maintaining pressure within the inflatable member to be the same or substantially the same as the blood pressure in the right atrium. Monitoring pressure may also be used to maintain the pressure to ensure the inflatable member is stabilized or anchored in place.

In any of the embodiments described herein, a monitored pressure (e.g., positive and/or negative pressure) may be used as a means to determine when puncture of the septal wall is complete. That is, a change in pressure may occur when the piercing member pierces all the way through the distal membrane or septal wall, which may be sensed by the pressure monitor(s) (e.g., gauge). For example, if positive fluid pressure within the first volume/chamber of the inflatable member is being monitored, piercing through the distal membrane can be associated with a decrease in fluid pressure as the fluid is allowed to flow out of the hole of the distal membrane. If a negative pressure within the first volume/chamber of the inflatable member is being monitored, piercing through the septal wall can be associated with an increase in pressure as the negative pressure is allowed to flow out of the hole of the septal wall. As described herein, the negative pressure may be turned off once puncture through the septal wall is complete to reduce/prevent the drain of blood out of the left atrium.

Any of the inflatable members herein may have one or more irrigation ports therein, but are still considered to be closed inflatable members.

After the FO has been pierced, the piercing member may be used as a guidewire for a subsequently delivered medical tool into the left atrium. Everything except the piercing member may be removed prior to subsequent tool delivery.

In some methods, the piercing member and the guide may be removed and the elongate member and inflatable member (and optionally the optic(s) if secured to the inflatable member) may be left in the patient. Another device may be advanced through the elongate member and into the inflatable member. In any of the methods herein, the inflatable member may be collapsed, and the inflatable member may be advanced through the FO opening and re-inflated in the left atrium. The inflatable member may then be used, for example only, to visualize one or more structures or anatomical regions, which may be part of a diagnostic procedure and/or therapeutic procedure. For example only, a subsequent tool may be advanced into and/or through the inflatable member, such as into the left atrial appendage.

1. A tissue piercing assembly configured to pierce a septal wall between a right atrium and a left atrium, comprising:
a cone-shaped member having inflatable walls and a closed inner chamber, wherein a closed distal region of the inner chamber includes a membrane;
a visualization member adapted to be positioned to have a field of view that includes at least a portion of the closed distal region; and
one or more of a piercing member guide and a piercing member disposed within the inflatable chamber, wherein the piercing member has a distal end that is configured to pierce the membrane at the closed distal region of the cone-shaped member.

2. The assembly of claim 1, wherein the inflatable wall comprises one or more second inflatable chambers disposed radially outward relative to a proximal region of the inflatable chamber, at least one of the one or more second inflatable chambers not in direct fluid communication with the closed inner chamber.

3. The assembly of claim 1, wherein the membrane has a lesser thickness than the inflatable walls.

4. The assembly of claim 1, wherein the closed distal region is adapted to have a planar or flattened configuration when the cone-shaped member is at least partially inflated.

5. The assembly of claim 1, wherein the closed distal region is less stiff than a proximal region of the cone-shaped member when the cone-shaped member is at least partially inflated.

6. The assembly of claim 5, wherein the proximal region includes at least one second inflatable chamber.

7. The assembly of claim 1, further comprising one or more controllers configured to control a first fluid pressure within the closed inner chamber and a second fluid pressure within the inflatable walls, wherein the first fluid pressure is different than the second fluid pressure.

8. The assembly of claim 1, wherein the closed distal region has a flattened configuration when the cone-shaped member is in an at least partially inflated configuration.

9. The assembly of claim 1, wherein the assembly includes the piercing member guide and the piercing member, the piercing member guide including a piercing member lumen sized and configured to receive the piercing member therethrough.

10. The assembly of claim 1, wherein the one or more of the piercing member guide and the piercing member is axially movable relative to the cone-shaped member.

11. The assembly of claim 1, wherein the assembly includes the piercing member guide, the piercing member guide having a sharpened distal end.

12. The assembly of claim 1, wherein the assembly includes the piercing member guide, the piercing member guide not having a sharpened distal end that it is not configured to pierce tissue.

13. The assembly of claim 1, wherein the visualization member includes an optical element secured relative to the one or more of the piercing member guide and the piercing member that is disposed within the cone-shaped chamber.

14. The assembly of claim 13, wherein the optical element is secured relative to the one or more of the piercing member guide and the piercing member such that the optical element is adapted to self-deploy to a deployed configuration.

15. (canceled)

16. The assembly of claim 1, wherein assembly includes the piercing member guide, wherein the visualization member is disposed within a visualization member receiving area of the piercing member guide when in a delivery state.

17-22. (canceled)

23. The assembly of claim 2, wherein the visualization member is secured within any of the one or more second inflatable chambers.

24-32. (canceled)

33. A method of piercing a fossa ovalis, comprising:
delivering a cone-shaped member having a closed distal region into a right atrium while the cone-shaped member is in a contracted configuration, the closed distal region comprising a membrane;
at least partially inflating an inflatable wall of the cone-shaped member to transition the cone-shaped member to an expanded configuration in the right atrium;
at least partially inflating a closed inner chamber of the cone-shaped member in the right atrium;
providing a field of view that includes the membrane and the fossa ovalis with a visualization member disposed within the closed inner chamber of the cone-shaped member; and
piercing the membrane of the cone-shaped member and the fossa ovalis with a piercing member to create an opening in the fossa ovalis.

34. The method of claim 33, wherein piercing the membrane and the fossa ovalis comprises advancing a piercing member through the membrane and the fossa ovalis.

35. The method of claim 33, wherein piercing the membrane comprises activating a RF electrode.

36. The method of claim 33, further comprising advancing a piercing member guide distally and into contact with an inner surface of the membrane.

37-118. (canceled)
